(19) United States
(12) Patent Application Publication
(10) Pub. No.: US 2022/0280943 A1
(43) Pub. Date: Sep. 8, 2022

(54) DIELECTROPHORETIC IMMOBILIZATION OF A PARTICLE IN PROXIMITY TO A CAVITY FOR INTERFACING

(71) Applicant: Mekonos, Inc., San Francisco, CA (US)

(72) Inventors: Mark A. Webb, Berkeley, CA (US); Marc S. Chooljian, Albany, CA (US)

(21) Appl. No.: 17/605,558

(22) PCT Filed: Apr. 22, 2020
(86) PCT No.: PCT/US2020/029387

§ 371 Date: Oct. 21, 2021

Related U.S. Application Data
(60) Provisional application No. 62/837,646, filed on Apr. 23, 2019.

(51) Int. Cl. B01L 3/00; B03C 5/00; C12M 1/42

(52) U.S. Cl. CPC B01L 3/502761; B03C 5/005; C12M 35/02; B01L 2200/0668; B01L 2300/0645; B01L 2400/0424

(57) ABSTRACT

An apparatus for immobilizing a particle in a fluid and a method for operating the apparatus are disclosed. The apparatus includes a membrane for separating a fluid from a compartment, one or more electrodes disposed proximate to the membrane, a counter-electrode, wherein the one or more electrodes and the counter-electrode are configured to generate a non-linear electric field across the one or more electrodes and the counter-electrode, and a power source for providing an alternating current (AC) across the one or more electrodes and the counter-electrode, thereby generating an oscillating non-linear electric field for immobilizing a particle suspended in the fluid that flows between the one or more electrodes and the counter-electrode. The membrane can have an opening to allow for mechanical manipulation of the particle that is immobilized with a sharp member configured to enter across the membrane from the compartment.

### BACKGROUND

Dielectrophoresis (DEP) is an electro-physical phenomenon that occurs when an electrically neutral, but polarizable, substance, such as a biological molecule or a cell, in a non-linear electric field experiences a force in the electric field gradient. This occurs because one side of the particle experiences a larger dipole force than the other due to the variation in electric field across the particle. The DEP force is nominally given by the following equation:

F_{DEP}=π∈ₘr³Re(f_{CM})∇|E|²

where r is the radius of the particle, ∈ₘ is the permittivity of the fluid, E is the electric field, and f_{CM} is the Clausius-Mossotti factor, a complex value that depends on the difference in permittivity between the fluid and the particle, and which determines if the DEP force will be positive or negative.

Based on the ability to trap and sort neutral particles or biological molecules in fluidic environments, DEP can be exploited, for example, for single-cell analyses in microfluidic-based applications. Using DEP in standard biochemical assays, for example, by applying DEP to isolate single cells for impedance or fluorescence characterization (or any non-contact evaluation technique) has been demonstrated in fluidic environments. However, using DEP to isolate single cells for direct manipulation of the cells presents additional challenges due to, for example and not limited to, the introduction of a probing tool for local manipulation of the cells in a fluidic and non-linear electric field environment. Therefore, there is a need for a novel system and technological platform that can employ DEP to isolate single cells for direct manipulation in a fluidic and non-linear electric field environment.

### SUMMARY

In accordance with various embodiments, an apparatus configured for immobilization of a particle is provided. The apparatus includes a membrane for separating a fluid from a compartment; one or more electrodes disposed proximate to the membrane; a counter-electrode, wherein the one or more electrodes and the counter-electrode are configured to generate a non-linear electric field across the one or more electrodes and the counter-electrode; and a power source for providing an alternating current (AC) across the one or more electrodes and the counter-electrode, thereby generating an oscillating non-linear electric field for immobilizing a particle suspended in the fluid that flows between the one or more electrodes and the counter-electrode.

In accordance with various embodiments, a method for operating an apparatus for immobilization of a particle is provided. The method includes providing a power source; providing a membrane configured for separating a fluid from a compartment; providing one or more electrodes disposed proximate to the membrane; providing a counter-electrode, wherein the one or more electrodes and the counter-electrode are configured to generate a non-linear electric field across the one or more electrodes and the counter-electrode; supplying, via the power source, an alternating current (AC) across the one or more electrodes and the counter-electrode, thereby generating an oscillating non-linear electric field; and immobilizing, via a dielectrophoretic force generated by the oscillating non-linear electric field, a particle suspended in the fluid that flows between the one or more electrodes and the counter-electrode.

In accordance with various embodiments, an apparatus configured for immobilization of a particle is provided. The apparatus includes one or more electrodes and a counter-electrode configured for generating a non-linear electric field for immobilizing a particle suspended in a fluid that flows between the one or more electrodes and the counter-electrode; and a membrane disposed proximate a surface of the one or more electrodes, the surface of the one or more electrodes distal the counter-electrode, wherein the membrane is configured for separating the fluid from a compartment, and has an opening configured to allow for insertion of a sharp member disposed in the compartment.

In accordance with various embodiments, a method for operating an apparatus for immobilization of a particle is provided. The method includes providing a power source; providing one or more electrodes and a counter-electrode configured for generating a non-linear electric field for immobilizing a particle suspended in a fluid that flows between the one or more electrodes and the counter-electrode; providing a membrane disposed proximate a surface of the one or more electrodes, the surface of the one or more electrodes distal the counter-electrode, wherein the membrane is configured for separating the fluid from a compartment, and has an opening configured to allow for insertion of a sharp member disposed in the compartment; supplying, via the power source, an alternating current (AC) across the one or more electrodes and the counter-electrode, thereby generating an oscillating non-linear electric field; and immobilizing, via a dielectrophoretic force generated by the oscillating non-linear electric field, a particle suspended in the fluid.

In accordance with various embodiments, a method for operating an apparatus for immobilization of a particle is provided. The method includes providing a power source; providing a membrane configured for separating a fluid from a compartment; providing a pair of electrodes disposed proximate a surface of the membrane, wherein the pair of electrodes is configured to generate a non-linear electric field across the electrodes; supplying, via the power source, an alternating current (AC) across the electrodes, thereby generating an oscillating non-linear electric field; and immobilizing, via a dielectrophoretic force generated by the oscillating non-linear electric field, a particle suspended in the fluid that flows between the electrodes. The method also includes providing a counter-electrode. The method also includes providing a third electrode disposed proximate the surface of the membrane.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIGS. 1A-1D show schematic views of an apparatus configured for immobilization of a particle, in accordance with various embodiments.

FIGS. 2A-2D show schematic illustrations of an apparatus configured for immobilization of a particle, in accordance with various embodiments.

FIGS. 3A-3D show schematic illustrations of an apparatus configured for interrogation of a particle, in accordance with various embodiments.

FIG. 4 shows a schematic illustration of an apparatus configured for location manipulation of a particle, in accordance with various embodiments.

FIGS. 5A-5D are various schematic views of the apparatus 400 configured for location manipulation of a particle, in accordance with various embodiments.

FIGS. 6A-6D illustrate various configurations of an apparatus configured for immobilization of a particle, in accordance with various embodiments.

FIGS. 7A-7C show schematic illustrations of various configurations of an apparatus configured for immobilization of a plurality of particles, in accordance with various embodiments.

FIG. 8 is a graphical diagram displaying simulation results for an apparatus for immobilization of a particle, in accordance with various embodiments.

FIG. 9 is a three-dimensional chart showing results of an analysis for an apparatus for immobilization of a particle, in accordance with various embodiments.

FIG. 10 is a flow chart for an example method of operating an apparatus for immobilization of a particle, in accordance with various embodiments.

FIG. 11 is a flow chart for an example method of operating an apparatus for immobilization of a particle, in accordance with various embodiments.

FIG. 12 is a flow chart for an example method of operating an apparatus for immobilization of a particle, in accordance with various embodiments.

### DETAILED DESCRIPTION

As described herein, the term “particle” refers to an object or a group of objects that individually or together have a physical property. The particle has a composition that can include mixtures, including, but not limited to living cells, viruses, oil droplets, liposomes, micelles, reverse micelles, protein aggregates, polymers, surfactant assemblies or their combination. The particle can be an individual, or a plurality of, cell (or cells), virus (or viruses), bacterium or bacteria, or any organism(s), alive or dead. The particle can be free floating in a fluid, e.g., suspended in the fluid, can be adherent, can change shape, can merge, can split apart, etc.

The term “pore” refers to an opening between two regions. The term “payload” includes any chemical compound, polymer, biological macromolecule, or combination. The term “signal” includes any electrical events, such as variations in voltage, current, frequency, phase, or duration that may comprise DC, AC, or a superposition of frequency components. The term “interference” refers to any electromagnetic disturbance that interrupts, obstructs, or otherwise degrades or limits the effective transmission or readout of a signal or signal component. The term “membrane” refers to any partition or physical barrier separating two regions. The term “interrogation” refers to activities such as, for example, material sampling, physical probing, sensing, payload delivery, interaction, physical touching, capillary wicking, and/or insertion.

The disclosure generally relates to an apparatus for local manipulation of neutral particles or biological molecules in a fluidic and non-linear electric field environment and various (e.g., microfluidics) applications thereof. In particular, the disclosure relates to an apparatus for dielectrophoresis-based (DEP-based) immobilization of biological objects, single cells or groups of cells in proximity to a compartment (or cavity) for local manipulation of the molecules or cells. In various implementations, the compartment or cavity can be filled with one of an aqueous fluid, an aqueous buffer, an organic solvent, a hydrophobic fluid, or a gas. In various implementations, the compartment can contain a fluid within the compartment that is immiscible with a fluid outside the compartment. In various implementations, the compartment can contain a non-aqueous fluid or microelectronics incompatible with an aqueous environment.

Further, the disclosure relates to an apparatus for local manipulation of an individual object or cell across a compartment via an interface with Micro-Electro-Mechanical System (MEMS) based structures and/or probing tools and/or electrodes for Nanopore Electroporation (NEP) applications. Suitable applications based on the technology disclosed herein includes in-situ biological interrogation, cellular engineering, single cell genomics, electrochemical and physical interrogation of biological samples (e.g., patch clamp or atomic force microscopy (AFM)), droplet microfluidics (e.g., sampling or microinjection of droplet fluid), and any other suitable applications. Suitable applications that the technology can be applied to include interrogation of discrete biologics, e.g., interrogation or probing of cells, living cells, viruses, oil droplets, liposomes, micelles, reverse micelles, protein aggregates, polymers, surfactant assemblies or their combination, etc.

The technology disclosed herein relates to coupling aqueous microfluidic environments with structures that can be in non-aqueous environments, e.g. electronics that can be in a non-conductive fluid or processes which can use hydrophobic solvents. The disclosed technology can offer local manipulation of isolated particles in a fluidic environment at scale, while allowing access from a compartment that contains sensitive MEMS components or electronics is disclosed. This can be done by coupling MEMS processes with microfluidic processes to allow for high-throughput processing and interrogation of suspended particles (the term particle or particles may refer to “biological object, objects or cells” and non-biological objects). In particular, the technology described herein relates to a high-throughput, DEP-based particle immobilization (trapping) apparatus that pins and immobilizes one or more particles in a fluid that flows adjacent to a membrane that separates the fluid from a compartment (isolated compartment or cavity) that contains electronic components, including MEMS structures. As described herein, in order to provide access from the cavity into the fluidic environment, one or more membrane openings (also refer to herein as “pores” or “micropores”) through the membrane can be used. For example, the openings can be used for providing access to one or more particles suspended in the fluid to be individually interfaced and/or interacted with individual MEMS/electrical structures residing across the membrane in the compartment. As described herein, the membrane can also be designed to maintain a stable liquid/gas interface or liquid-liquid interface between two immiscible fluids using fluid dynamic strategies that include, but are not limited to, surface patterning via hydrophobic or hydrophilic coatings, and/or pressure control of both fluid media on either side of the membrane. This interface can also be controlled to intentionally move fluid into or out of the cavity via modulation of surface energy via electrostatics, by pressurizing or depressurizing the cavity, or by changing the size or shape of the pore (e.g. by inserting a hollow microneedle into the pore to decrease the effective capillary radius).

By providing a platform to interface the DEP-mediated particle immobilization technique (e.g., a trapping technique) with highly local manipulation of individual biological molecules or cells at the single-cell level (e.g., single cell resolution), a highly controllable approach to extraction of genetic materials and/or delivery of drug molecules into individual cells, for example, can be achieved, not just for single cells, but in a high-throughput, reliable, and reproducible fashion.

As described herein, various implementations of an apparatus for immobilizing a particle in fluid is described. In various implementations, the apparatus includes a membrane for separating a fluid, for example, in a microfluidic channel, from a compartment. In various implementations, the apparatus also includes one or more electrodes disposed on the membrane away from the compartment and a counter-electrode having a dissimilar surface area than the one or more electrodes. In various implementations, the one or more electrodes and the counter-electrode (also referred to herein as “DEP electrodes”) are configured to generate a non-linear electric field across the one or more electrodes and the counter-electrode. In various implementations, the apparatus also includes an electrical input and output source for providing and sensing a signal across the one or more electrodes and/or the counter-electrode. In various implementations, the signal is an AC voltage for generating an oscillating non-linear electric field for immobilizing a particle suspended in the fluid that flows between the one or more electrodes and the counter-electrode.

In various implementations of the apparatus, the membrane has an opening through which to allow for mechanical manipulation of the particle that is immobilized. In various implementations, the mechanical manipulation includes probing the particle with a sharp member configured to enter across the membrane from the compartment. In various implementations, the sharp member is a MEMS structure or a Nano-Electro-Mechanical System (NEMS) structure. In various implementations, the sharp member is a needle, a pillar or a hollow tube.

In various implementations, the disclosed technology relates to an apparatus having a microfluidics membrane hybrid architecture that is tailored for optimum interrogation of discrete objects (e.g., discrete globular objects) suspended in a fluid medium. Using the apparatus, the globular objects can be spatially confined using DEP in proximity to the membrane, including a porous membrane. In various implementations, the pores in the membrane are geometrically and chemically optimized/tailored to prevent fluid exchange across the membrane. Applications of the apparatus can include interrogating discrete biological systems within a fluidic environment by an external probe. Moreover, the technology related to the microfluidics membrane hybrid architecture described herein can be integrated into a larger device architecture via typical MEMS fabrication methods. For example, the external probe can be fabricated, via MEMS fabrication methods, and disposed in the compartment.

In various implementations, the apparatus includes an array of electrodes (or the array of one or more electrodes, e.g., a pair of electrodes, a set of three electrodes, a set of four electrodes, and so on) co-localized with pores (e.g., opening 125, 225a-d, etc.), allowing access to trapped particles from a cavity. In various implementations, the pores are made to be hydrophobic by a chemical treatment coating the interior walls of the pores. In various implementations, the edge surface of the pores on either side of the membrane and/or pore interior are coated/chemically functionalized with a range of material classes including, for example, any small molecule, proteins, peptides, peptoids, polymers, or inorganic materials listed above in any suitable combination. Some examples of surface chemistries and their functionalities are included herein. In accordance with various embodiments, coatings of the interior of the pore and/or one side of the membrane can include a hydrophobic material, such as a hydrophobic organosilane, e.g. a fluorosilane, in order to prevent leakage of aqueous solution through the pore. In accordance with various embodiments, a surface can be coated to discourage cell adhesion, using a chemical such as for example, but not limited to, a poloxamer or poly(2-hydroxyethyl methacrylate) or any suitable protein blocking solution, such as for example, bovine serum albumin, in order to prevent nonspecific cell adhesion away from trapping sites, for example, approximate the opening or pore. Some examples of surface coatings may include, for example, biological or organic materials, such as proteins, peptides, polymers, hydrocarbon chains of varying lengths, any combination of which can be used for preventing cell adhesion as well as payload/analyte adhesion prevention. In accordance with various embodiments, such surface coatings may be used for preventing molecular payload adhesion, particularly with respect to molecular payload that is disposed on a sharp member or needle. In accordance with various embodiments, a coating on one side of the membrane with a hydrophilic material such as hyaluronic acid, titanium oxide, polyethylene glycol, etc. in order to ensure efficient wetting of those surfaces and prevent outflow flow of hydrophobic material from the opening. In accordance with various embodiments, any combination of the aforementioned approaches can be employed in order to separate hydrophobic and hydrophilic fluids in separate openings, pores, or cavities.

The various implementations disclosed herein represent a unique capability for high-volume trapping of biological objects and/or cells for characterization, sampling, payload delivery, or modification via techniques, such as, for example, electrochemical, impedometric, optical methods, and MEMS-based cellular manipulation in a fluidic environment. As described herein, physical and material properties and parameters, such as, for example, the size and hydrophobicity of the pore (or opening), size of the electrode, conductivity of the fluid medium, and operating frequency of the electrodes can be optimized based on the application and the biological objects or cells to be interrogated. In various implementations of the technology described herein, the apparatus can be configured for selective release of cells after trapping/capture, and probing/interrogation/manipulation.

Moreover, according the various implementations as described herein, the apparatus can also be optimized by exploiting the dielectrophoretic (DEP) force. For example, since the DEP force generated is proportional to the square of the field gradient according to the DEP equation described above, a highly non-linear electric field can be generated across the one or more electrodes and the counter-electrode. In various implementations, by applying an alternating current (AC) between one or more electrodes with a geometry that creates a large electric field gradient via size difference and/or proximity, a confined highly non-linear electric field can be generated to act on a biological object or cell, and immobilize it in the trapping area. For example, if the one or more electrodes, e.g., a pair of electrodes, are arranged around an opening, the DEP force can be tuned to trap the object between the electrodes at the opening. In addition, if the wall of the opening in the electrode is coated with a hydrophobic material, the contact angle of the coated inner wall of the opening can relate to the capillary pressure of the fluid via the following equation:

$$Δ p = - \frac{2 γ cos ( θ )}{r}$$

where r is the radius of the opening, y is the surface tension (approximately 72.75 mN/m for water and air) and θ is the contact angle. Conventionally, a contact angle θ of above 90 represents a hydrophobic material while a contact angle below 90 represents a hydrophilic material. By increasing the contact angle θ to around 130 degrees by applying, for example, a hydrophobic silane coating, the capillary pressure for an air-water interface reaches 40-60 kPa with a relatively large opening of about 4 μm or 5 μm. As described herein, a hydrophobic coating on the inner wall of the opening can prevent fluid from flowing through the opening from the aqueous side into an air-filled compartment that can contain MEMS or other electronic components. The same principle holds for other types of fluid phase separation across the membrane, depending on whether the aqueous or non-aqueous side of the membrane is at higher or lower pressure the pore can be patterned with a hydrophobic or hydrophilic surface treatment respectively. Therefore, the apparatus having one or more electrodes and a counter-electrode arranged in such a way to produce a nonlinear electric field can be configured to trap, immobilize or confine a biological object or a cell in a fluid and to be probed, via an opening, by a MEMS structure that resides in the compartment without compromising any fluid exposure to the sensitive electronic components. In various implementations, the apparatus has one or more electrodes and a counter-electrode that are of the same or substantially similar size can be configured to generate a highly non-linear electric field in order to trap, immobilize or confine a biological object or a cell in a fluid and to be probed, via an opening, by a MEMS structure that resides in the compartment without compromising any fluid exposure to the sensitive electronic components. In various implementations, each of the trapping sites e.g., an opening or a pore, can include an electrode, two electrodes, three electrodes, four electrodes, and so on. In various implementations, additional electrodes can be configured for impedance sensing in the presence of an object, for example, a particle or a cell. Furthermore, since the fabrication of the apparatus can be done using well-established MEMS processing techniques and highly reliable and reproducible methods based on photolithography, the method of manufacturing the apparatus is scalable, and thus allowed for parallel immobilization and interrogation of biological objects or cells at clinically relevant quantities.

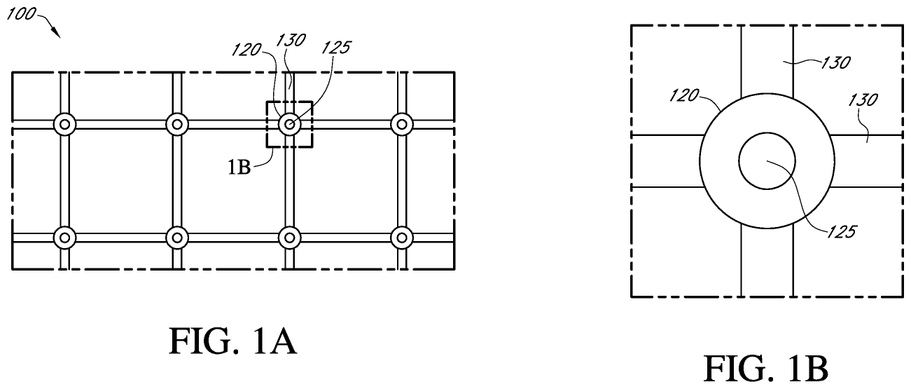

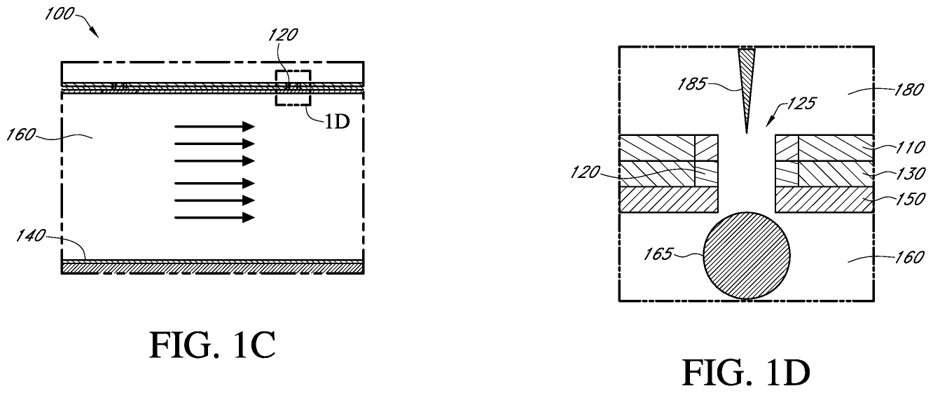

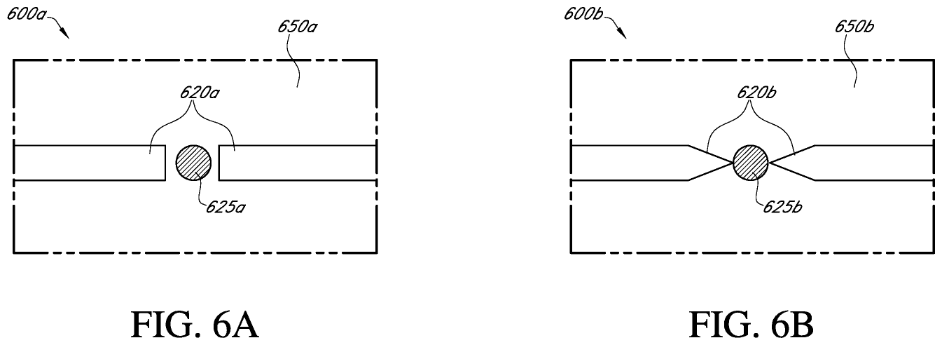

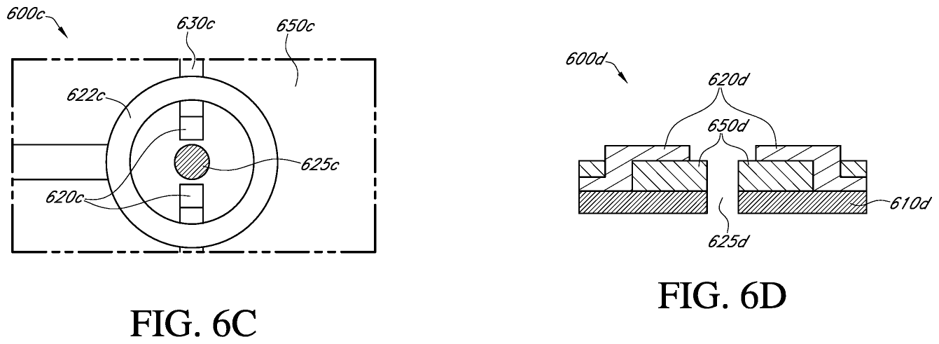

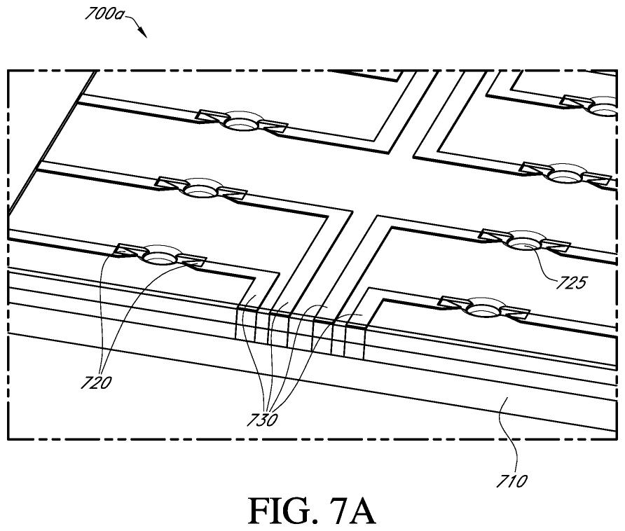

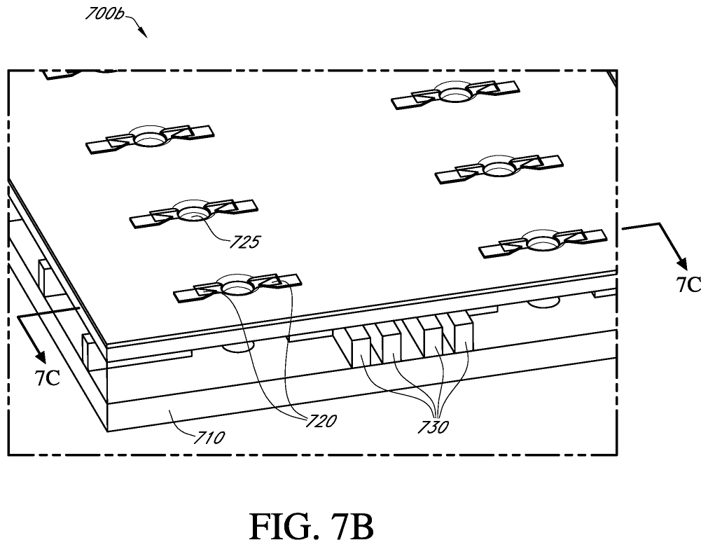

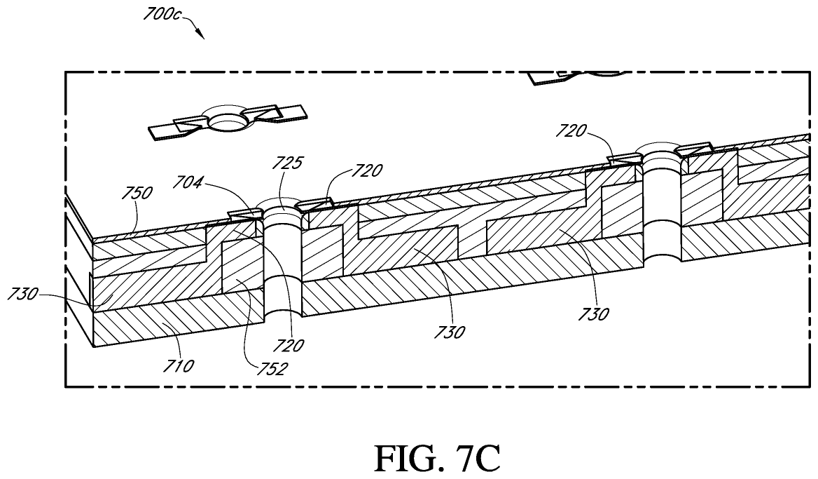

FIGS. 1A-1D show schematic views of an apparatus for immobilization of a particle, according to various implementations as disclosed herein. FIG. 1A shows a schematic top view of an example apparatus 100, in accordance with various embodiments. As shown in FIG. 1A, the apparatus 100 includes an opening 125 (also referred to herein as “pore”), a plurality of electrodes 120 and one or more interconnects 130. For example, the plurality of electrodes 120, as illustrated, can include a plurality of individual disparate electrode surface areas formed in an array or a grid. Although the electrode 120 is illustrated as a ring or circular electrode, the electrode 120 can be a pair of electrodes 620a, 620b, 620c, 620d, 720 as shown and described with respect to FIGS. 6A-6D and 7A-7C, or any number of sets of electrodes disposed proximate the opening 125, in accordance with various embodiments. Accordingly, the physical, chemical, material parameters as described further below with respect to the electrode 120 can be applicable to any of the pair of electrodes 620a, 620b, 620c, 620d, 720 as shown and described with respect to FIGS. 6A-6D and 7A-7C.

In various implementations, the electrode 120 has a thickness between about 1 nm to about 50 μm. In various implementations, the electrode 120 has a thickness between about 10 nm to about 5 μm, about 10 nm to about 10 μm, about 10 nm to about 5 μm, about 100 nm to about 4 μm, about 300 nm to about 3 μm, about 400 nm to about 5 μm, about 500 nm to about 5 μm, inclusive of any thickness ranges therebetween.

In various implementations, the electrode 120 includes at least one of a transparent conducting material or a doped semiconducting material with sufficient electrochemical stability. In various implementations, the transparent conducting material includes indium tin oxide, graphene, doped graphene, a conducting polymer, or a thin metal layer.

As shown in FIG. 1A, in various implementations, each of the plurality of electrodes 120 (referring to an array of electrodes 120) has an opening 125. In various implementations, some of the plurality of electrodes 120 have an opening 125 and some electrodes 120 do not have an opening 125. In various implementations, the electrodes 120 that have an opening 125 and the electrodes 120 that do not have an opening 125 are strategically arranged based on the application of the apparatus 100.

In various implementations, the opening 125 has a size (also referred to herein as a diameter if circular or a lateral dimension if any non-circular geometry) between about 0.1 nm to about 1 mm. In various implementations, the opening 125 has a size between about 1 nm to about 100 nm, about 100 nm to about 1 μm, about 1 μm to about 10 μm, about 100 nm to about 25 μm, about 1 μm to about 100 μm, or about 1 μm to about 50 μm, inclusive of any size ranges therebetween.

In various implementations, the electrodes 120 in the plurality of electrodes 120 have an electrode-to-electrode separation distance between two adjacent electrodes from about 1 μm to about 5 mm, from about 1 μm to about 1 mm, from about 10 μm to about 500 μm, or from about 10 μm to about 1 mm, inclusive of any separation distance ranges therebetween.

In various implementations, the electrode 120 and the one or more interconnects 130 include the same material. In various implementations, the one or more interconnects 130 includes at least one of a transparent conducting material or a doped semiconducting material with sufficient electrochemical stability. In various implementations, the transparent conducting material includes indium tin oxide, metal nanowire mesh, graphene, a doped graphene, a conducting polymer, a thin metal layer, an atomic-layer metal film, or any other suitable transparent conductor.

FIG. 1B shows a zoomed-in schematic view of one of the electrodes 120 of the apparatus 100. In various implementations, the apparatus 100 includes one electrode 120. As shown in FIGS. 1A and 1B, the plurality of electrodes 120 are interconnected to each other via one or more interconnects 130 in a grid or in an array. In various implementations, the plurality of electrodes 120 are interconnected to each other within a group that can include any number of electrodes 120, and the apparatus 100 can include any number of groups of electrodes 120.

FIG. 1C shows a cross-sectional view (orthogonal to the view of FIG. 1B) of the apparatus 100, according to various implementations. As shown in FIG. 1C, the apparatus 100 includes the plurality of electrodes 120 and a counter-electrode 140. In accordance with various embodiments, each electrode 120 in the plurality of electrodes 120 can be a pair of electrodes 620a, 620b, 620c, 620d, 720 as shown and described with respect to FIGS. 6A-6D and 7A-7C, or any number of sets of electrodes disposed proximate the opening 125. In various implementations, the counter-electrode 140 is a plane electrode that spans across a portion, a substantial portion, almost an entirety, or an entirety of the apparatus 100. For example, the counter-electrode 140 can be bigger than each of the plurality of electrodes 120. For example, the counter-electrode 140 can have a surface area that is bigger than a surface area of each of the individual electrodes 120. In various implementations, the ratio of the surface area between the counter-electrode 140 and an electrode 120 can be about 1:1, 1.1:1, 2:1, 5:1, 10:1, 50:1, 100:1, 1 million: 1, or any suitable ratios therebetween.

In various implementations, the electrode 120 and the counter-electrode 140 have the same or substantially similar in size. In various implementations, the electrode 120 and the counter-electrode 140 are disposed on the same plane.

As illustrated in FIG. 1C, the plurality of electrodes 120 and the counter-electrode 140 are configured to receive a fluid (indicated as parallel arrows in FIG. 1C) that flows in a channel 160 between the plurality of electrodes 120 and the counter-electrode 140. In various implementations, the fluid that flows in the channel 160 can include, for example, but not limited to, an aqueous fluid, an aqueous buffer, an organic solvent, a hydrophobic fluid, or a gas.

In various implementations, the fluid flows in the channel 160 at a flow rate between 0 to 10 mL/s. In various implementations, the fluid is static and, therefore, has minimal to no flow rate. In various implementations, the fluid flows from about 0.001 mL/s to about 0.1 mL/s, about 0.01 mL/s to about 1 mL/s, or about 0.1 mL/s to about 10 mL/s, inclusive of any flow rate ranges therebetween.

FIG. 1D shows a zoomed-in cross-sectional view of one of the plurality of electrodes 120 of the apparatus 100. As shown in FIG. 1D, the apparatus 100 includes a membrane 110, the electrode 120, an interconnect 130, and a passivation layer 150. In various implementations, the membrane 110 includes an electrically insulating material. In various implementations, the membrane 110 includes an electrically insulating material, including, but not limited to silicon nitride, silicon oxide, a metal oxide, a carbide (such as, for example, SiCOH), a ceramic (such as, for example, alumina), and a polymer. In various implementations, the membrane 110 includes an electrically conducting material, such as a metal or a doped semiconductor material. In various implementations, the membrane 110 can be a single layer or a composite layer having a multilayer stack that includes any of the aforementioned materials.

In various implementations, the wall forming the channel 160 comprises a channel material that can include, for example but not limited to, silicon, glass, plastic, or various elastomers such as, for example, poly(dimethyl siloxane) (PDMS), which can be used as structural materials for the fluidic layer. In various implementations, the channel 160 has dimensions from about 1 nm to about 1 cm, from about 100 nm to about 100 mm, from about 200 nm to about 1 mm, or from about 200 nm to about 500 μm, inclusive of any dimensions therebetween. In various implementations, the height of the channel 160 is set by the particle size being probed and in order to avoid clogging should be at least twice the diameter of the particle.

In various implementations, the membrane 110 has a thickness between about 10 nm to about 1 cm. In various implementations, the membrane has a thickness between about 10 nm to about 5 mm, between about 10 nm to about 1 mm, between about 10 nm to about 100 μm, about 50 nm to about 10 μm, about 50 nm to about 5 μm, about 100 nm to about 10 μm, about 100 nm to about 5 μm, or about 100 nm to about 2 μm, inclusive of any thickness ranges therebetween. In various implementations, the membrane 110 or any layer of material comprising the membrane can be patterned.

FIG. 1D also shows a particle 165 that is suspended in the fluid that flows in the channel 160. In various implementations, the particle 165 can include various types of particulate material or globular materials, including, but not limited to, any biological objects, cells, or non-biological objects. In various implementations, the particle 165 can include a biological organism, a biological structure, a cell, a living cell, viruses, oil droplets, liposomes, micelles, reverse micelles, protein aggregates, polymers, surfactant assemblies, a vesicle, a micro-vesicle, a protein, a molecule, a microdroplet, or a non-biological particulate matter.

In various implementations, the particle 165 can have a size between about 1 nm to about 1 mm. In various implementations, the particle 165 can have a size between about 10 nm to about 500 μm, about 50 nm to about 200 μm, about 200 nm to about 100 μm, about 300 nm to about 50 μm, about 100 nm to about 200 μm, about 100 nm to about 100 μm, or about 200 nm to about 50 μm, inclusive of any size ranges therebetween.

As shown in FIG. 1D, according to various implementations, the membrane 110 is configured to separate the fluid from entering a compartment 180. FIG. 1D also shows the opening 125 of the apparatus 100. As shown in FIG. 1D, the opening 125 extends through the membrane 110 and the electrode 120. In various implementations, the opening 125 extends through the membrane 110, the electrode 120, and the passivation layer 150. In various implementations, the opening 125 may also serve as a capillary valve to isolate two fluid phases across the membrane 110 if the operation of the device requires more than one fluid phase (such as an ionic buffer and air or aqueous and organic solvents). The zoomed-in cross-sectional view of FIG. 1D also shows a sharp member 185 disposed in the compartment 180 near the membrane 110.

In various implementations, the sharp member 185 is configured to move within the opening 125, and move through the membrane 110, the electrode 120, and the passivation layer 150. According to various implementations, the opening 125 allows for mechanical manipulation of the particle 165 that is immobilized. In various implementations, the mechanical manipulation includes probing, inserting, penetrating, electroporating, sensing, depositing material, sampling material, or otherwise manipulating the particle 165 with the sharp member 185 configured to enter across the membrane 110, the electrode 120, and/or the passivation layer 150. In various implementations, the mechanical manipulation is conducted by the sharp member 185. In various implementations, the sharp member 185 enters from the compartment 180 where the sharp member 185 resides before the movement, e.g., before actuation of the sharp member 185 along the longitudinal axis, e.g., vertically downward, as shown in FIG. 1D. In various implementations, the sharp member 185 can be a needle, a pillar, a hollow tube, nano-needle or a micro-needle having a length between about 10 nm to about 50 μm. In various implementations, the sharp member 185 is manufactured or fabricated via Micro-Electro-Mechanical System (MEMS) methods or Nano-Electro-Mechanical System (NEMS) methods. In accordance with various embodiments, the compartment 180 includes a MEMS structure or a NEMS structure, including the sharp member 185.

In various implementations, the sharp member 185 can be configured to operate as a third electrode in the form of a probe across the membrane 110. This third electrode probe may be biased with a DC or AC signal for sensing or actuation, for instance with a pulsed DC signal for Nanopore Electroporation (NEP) applications or with a low-power AC signal of a separate frequency for the purpose of measuring impedance. In various implementations, the DEP electrodes themselves may also carry a separate superposed AC or DC signal chosen to be easily isolated from the DEP signal via downstream filtering.

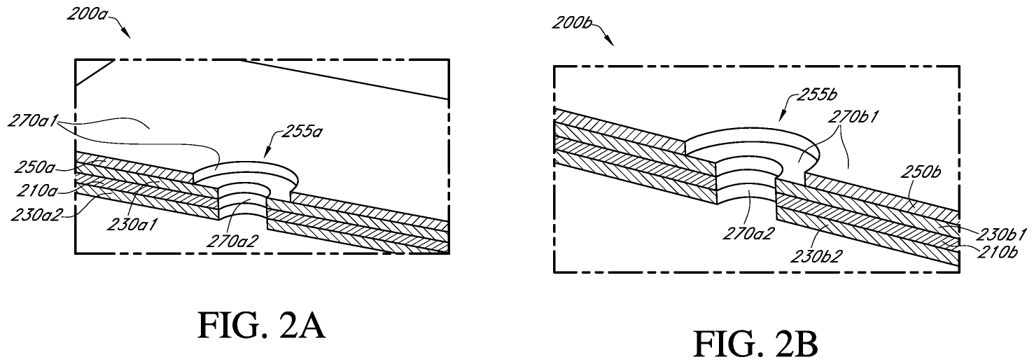

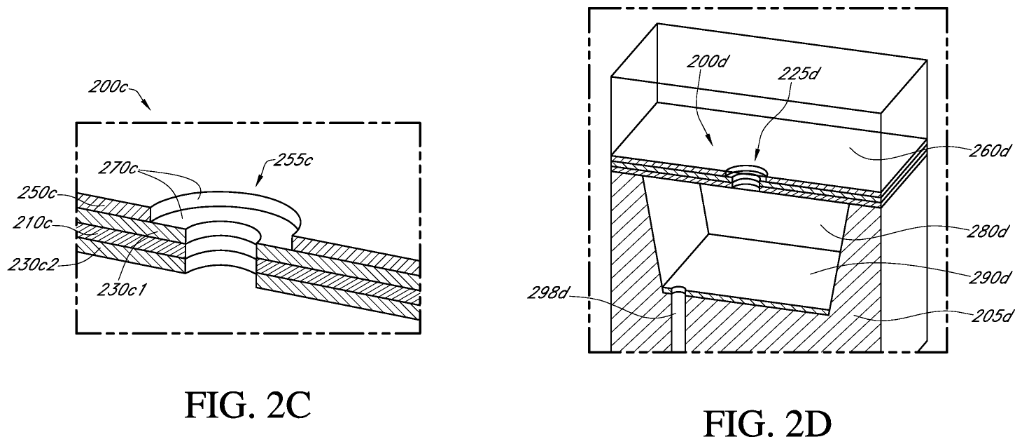

In various implementations, a wall of the opening 125 has a hydrophobic coating or a hydrophilic coating. In various implementations, the opening 125 is made to be hydrophobic by a chemical treatment coating the interior walls of the opening 125. In various implementations, the edge surface of the opening 125 on either side of the membrane and/or an inside of the wall (inner wall) of the opening 125 (also referred to herein as “pore interior”) are coated/chemically functionalized with a range of material classes including, for example, any small molecule, proteins, peptides, peptoids, polymers, or inorganic materials listed above in any suitable combination. Various details of the coatings are provided in detail with respect to FIGS. 2A-2D.

In accordance with various embodiments, the hydrophobic coating or hydrophilic coating are disposed (or deposited) on wall of the membrane 110 and/or the electrode 120 to prevent the fluid from entering into the compartment. In various implementations, the coating is chemically and covalently attached to the relevant surfaces. In various implementations, the hydrophobic coating can include a variety of classes such as azides, organosilanes, or fluorocarbons. In various implementations, the hydrophilic coating can include a range of material classes including any small molecule, proteins, peptides, peptoids, polymers, or inorganic materials. In various implementations, the wall of the opening 125 has a combination of patterned hydrophilic and hydrophobic coatings.

In various implementations, the hydrophobic coating has a contact angle between about 95° and about 165°. In various implementations, the hydrophobic coating has a contact angle between about 100° and about 165°, about 105° and about 165°, about 110° and about 165°, about 120° and about 165°, about 95° and about 150°, about 95° and about 140°, or about 95° and about 130°, inclusive of any contact angle ranges therebetween.

In various implementations, the hydrophilic coating has a contact angle between about 20° and about 80°. In various implementations, the hydrophilic coating has a contact angle between about 25° and about 80°, about 30° and about 80°, about 35° and about 80°, about 40° and about 80°, about 20° and about 70°, about 20° and about 60°, or about 20° and about 50°, inclusive of any contact angle ranges therebetween.

According to various implementations, a power source (not shown) can be electrically connected to the plurality of electrodes 120 and the counter-electrode 140 to provide an alternating current (AC) across the plurality of electrodes 120 and the counter-electrode 140 to generate an oscillating non-linear electric field for immobilizing (or trapping) the particle 165 suspended in the fluid that flows between the plurality of electrodes 120 and the counter-electrode 140. In various implementations, an in-plane electric field with multiple electrodes can be applied to induce a local field minimum for alternate DEP field. In various implementations, one or more AC or DC signals may be superposed on the DEP actuation signal for applications including impedance sensing, electrowetting, or electroporation.

In various implementations, the AC across the plurality of electrodes 120 (electrode 120 if a single electrode or a pair of electrodes, such as 620a, 620b, 620c, 620d, 720) and the counter-electrode 140 is supplied at a voltage between about 1 mV and about 300 V. In various implementations, the AC across the plurality of electrodes 120 and the counter-electrode 140 is supplied at a voltage between about 5 mV and about 50 V between about 5 mV and about 20 V, about 250 mV and about 5 V, about 500 mV and about 50 V, about 750 mV and about 50 V, about 1 V and about 50 V, about 5 V and about 50 V, about 10 V and about 50 V, about 250 mV and about 40 V, about 250 mV and about 30 V, about 250 mV and about 20 V, about 250 mV and about 10 V, about 250 mV and about 8 V, about 250 mV and about 6 V, about 250 mV and about 5 V, about 500 mV and about 5 V, or about 1 V and about 5 V, inclusive of any voltage ranges therebetween. In various implementations, the AC across the plurality of electrodes 120 (electrode 120 if a single electrode) and the counter-electrode 140 is supplied at a voltage between about 1 mV and about 20 V, between about 1 mV and about 10 V, between about 1 mV and about 8V, between about 1 mV and about 6 V, between about 1 mV and about 5 V, between about 1 mV and about 4 V, between about 1 mV and about 3 V, between about 1 mV and about 2 V, between about 1 mV and about 1 V, between about 1 mV and about 750 mV, between about 1 mV and about 500 mV, between about 1 mV and about 250 mV, between about 1 mV and about 200 mV, between about 1 mV and about 150 mV, between about 1 mV and about 100 mV, between about 1 mV and about 50 mV, inclusive of any ranges therebetween.

In various implementations, the AC across the plurality of electrodes 120 (electrode 120 if a single electrode or a pair of electrodes, such as 620a, 620b, 620c, 620d, 720) and the counter-electrode 140 is supplied at an oscillating frequency between about 1 Hz and about 1 THz. In various implementations, the AC across the plurality of electrodes 120 and the counter-electrode 140 is supplied at an oscillating frequency between about 10 Hz and about 100 GHz, about 10 Hz and about 10 GHz, about 100 Hz and about 10 GHz, about 1 kHz and about 1 GHz, about 10 kHz and about 1 GHz, about 100 kHz and about 1 GHz, about 500 kHz and about 1 GHz, about 1 MHz and about 1 GHz, about 10 MHz and about 1 GHz, about 100 MHz and about 1 GHz, about 10 kHz and about 500 MHz, about 10 kHz and about 100 MHz, about 10 kHz and about 50 MHz, about 10 kHz and about 30 MHz, about 10 kHz and about 20 MHz, about 10 kHz and about 10 MHz, about 100 kHz and about 10 MHz, or about 500 kHz and about 10 MHz, or about 1 MHz and about 10 MHz, inclusive of any frequency ranges therebetween.

In various implementations, a direct current (DC) is applied across the plurality of electrodes 120 (electrode 120 if a single electrode or a pair of electrodes, such as 620a, 620b, 620c, 620d, 720) and the counter-electrode 140. In various implementations, the DC and AC can be superimposed when applied a current across the plurality of electrodes 120 (electrode 120 if a single electrode or a pair of electrodes, such as 620a, 620b, 620c, 620d, 720) and the counter-electrode 140.

In various implementations, the plurality of electrodes 120 and the counter-electrode 140 can be individually addressed, addressed in groups, or electrically short-circuited (e.g., shorted) together. In various implementations, each in the pair of electrodes, such as 620a, 620b, 620c, 620d, 720 can be individually addressed, addressed in groups, or electrically short-circuited (e.g., shorted) together. For example, the AC can be supplied to each of the plurality of electrodes 120 and the counter-electrode 140 individually, or in groups. For example, the plurality of electrodes 120 and the counter-electrode 140 can be shorted for some of the plurality of electrodes 120 and the counter-electrode 140, and not the other electrodes 120 in the plurality of electrodes 120 and the counter-electrode 140. As such, any combination or configuration of arrangements between the plurality of electrodes 120 and the counter-electrode 140 can be implemented for the apparatus 100.

FIGS. 2A-2D show schematic illustrations of an apparatus configured for immobilization of a particle, in accordance with various embodiments. FIGS. 2A-2D illustrate various structural configurations of an apparatus, where the configurations illustrate, for example, but not limited to, specific layer arrangements, placement and type of coatings, such as hydrophobic or hydrophilic coatings. The configurations shown in FIGS. 2A, 2B, 2C, and 2D are non-limiting examples, and thus, any desired structural configurations in addition to the illustrations can be employed to perform immobilization and/or interrogation of a particle, in accordance with various embodiments.

FIG. 2A illustrates a cross-sectional view of an apparatus 200a, in accordance with various embodiments. As shown in FIG. 2A, the apparatus 200a includes a membrane 210a, a metal layer 230a1, a passivation layer 250a, and another metal layer 230a2 that are stacked on one another, and includes an opening 225a. The apparatus 200a also includes a coating 270a1 disposed on the exposed surface of the membrane 210a and a coating 270a2 disposed on the inside of the wall (inner wall) of the opening 225a, in accordance with various embodiments. As shown in FIG. 2A, the coating 270a1 and the coating 270a2 are the same coating. In accordance with various embodiments, the coatings 270a1 and 270a2 can include the same pattern or different patterns.

FIG. 2B illustrates a cross-sectional view of an apparatus 200b, in accordance with various embodiments. As shown in FIG. 2B, the apparatus 200b includes a membrane 210b, a metal layer 230b1, a passivation layer 250b, and another metal layer 230b2 that are stacked on one another, and includes an opening 225b. The apparatus 200b includes a coating 270b1 disposed on the exposed surface of the membrane 210b and a coating 270b2 disposed on the inside of the wall of the opening 225b, in accordance with various embodiments. As shown in FIG. 2B, the coating 270b1 and the coating 270a2 are different coatings. In accordance with various embodiments, the coatings 270b1 and 270b2 can include the same pattern or different patterns.

FIG. 2C illustrates a cross-sectional view of an apparatus 200c, in accordance with various embodiments. As shown in FIG. 2C, the apparatus 200c includes a membrane 210c, a metal layer 230c1, a passivation layer 250c, and another metal layer 230c2 that are stacked on one another, and includes an opening 225c. The apparatus 200c includes a coating 270c disposed on the inside of the wall of the opening 225b, and does not include a coating on the exposed surface of the membrane 210c, in accordance with various embodiments. In accordance with various embodiments, the coating 270c can include a pattern.

In accordance with various embodiments, the membranes 210a, 210b, and 210c can be the same or substantially similar to the membrane 110 as described with respect to FIG. 1D, unless stated otherwise, and therefore, will not be described in full detail. In various implementations, the membranes 210a, 210b, and 210c can include an electrically insulating material. In various implementations, the membranes 210a, 210b, and 210c can include an electrically insulating material, including, but not limited to silicon nitride, silicon oxide, a metal oxide, a carbide (such as, for example, SiCOH), a ceramic, such as, alumina, and a polymer. In various implementations, the membranes 210a, 210b, and 210c can include an electrically conducting material, such as a metal or a doped semiconductor material. In various implementations, the membranes 210a, 210b, and 210c can be a single layer or a composite layer having a multilayer stack that includes any of the aforementioned materials.

In various implementations, the membranes 210a, 210b, and 210c can have a thickness between about 10 nm to about 1 cm. In various implementations, the membranes 210a, 210b, and 210c can have a thickness between about 10 nm to about 5 mm, between about 10 nm to about 1 mm, between about 10 nm to about 100 μm, about 50 nm to about 10 μm, about 50 nm to about 5 μm, about 100 nm to about 10 μm, about 100 nm to about 5 μm, or about 100 nm to about 2 μm, inclusive of any thickness ranges therebetween.

In accordance with various embodiments, the metal layers 230a1, 230a2, 230b1, 230b2, 230c1, and 230c2 can be the same or substantially similar to the electrode(s) 120 and/or interconnect 130 as described with respect to FIGS. 1A-1D, unless stated otherwise, and therefore, will not be described in full detail. In accordance with various embodiments, the metal layers 230a1, 230b1, and 230c1 can be an electrode layer that can include for example, the electrode 120 or the electrodes 620a, 620b, 620c, 620d, 720). In accordance with various embodiments, the metal layers 230a1, 230b1, and 230c1 can be the interconnect layers 130 or 730. In accordance with various embodiments, the metal layers 230a2, 230b2, and 230c2 can be the interconnect layers 130 or 730, or an electrode layer that can be configured to use with a sharp member (e.g., 185, 385a-d, etc.), for sensing (as a sensing electrode), as an NEP electrode, or as a metal shielding electrode.

In accordance with various embodiments, the passivation layers 250a, 250b, and 250c can be the same or substantially similar to the passivation layer 150 as described with respect to FIG. 1D, unless stated otherwise, and therefore, will not be described in full detail.

In accordance with various embodiments, the coatings 270a1, 270a2, 270b1, 270b2, and 270c can be the same or substantially similar to the coating as described with respect to FIG. 1D, unless stated otherwise, and therefore, will not be described in full detail. In various implementations, each the coatings 270a1, 270a2, 270b1, 270b2, and 270c can be a hydrophobic coating or a hydrophilic coating. The hydrophobic coating or hydrophilic coating are disposed (or deposited) on the exposed surface of each of the membranes 210a and 210b, and/or on the inside of the wall (inner wall) of the openings 225a, 225b, and 225c to prevent a fluid from entering across the respective openings 225a, 225b, and 225c. In various implementations, the coatings 270a1, 270a2, 270b1, 270b2, and 270c are chemically and covalently attached to the relevant surfaces. In various implementations, the hydrophobic coating can include a variety of classes such as azides, organosilanes, or fluorocarbons. In various implementations, the hydrophilic coating can include a range of material classes including any small molecule, proteins, peptides, peptoids, polymers, or inorganic materials. In various implementations, the wall of each of the openings 225a, 225b, and 225c has a combination of patterned hydrophilic and hydrophobic coatings.

In various implementations, the hydrophobic coating of each the coatings 270a1, 270a2, 270b1, 270b2, and 270c can have a contact angle between about 95° and about 165°. In various implementations, the hydrophobic coating has a contact angle between about 100° and about 165°, about 105° and about 165°, about 110° and about 165°, about 120° and about 165°, about 95° and about 150°, about 95° and about 140°, or about 95° and about 130°, inclusive of any contact angle ranges therebetween.

In various implementations, the hydrophilic coating of each the coatings 270a1, 270a2, 270b1, 270b2, and 270c can have a contact angle between about 20° and about 80°. In various implementations, the hydrophilic coating has a contact angle between about 25° and about 80°, about 30° and about 80°, about 35° and about 80°, about 40° and about 80°, about 20° and about 70°, about 20° and about 60°, or about 20° and about 50°, inclusive of any contact angle ranges therebetween.

FIG. 2D illustrates a cross-sectional view of an apparatus 200d, in accordance with various embodiments. In accordance with various embodiments, the apparatus 200d can be the same or substantially similar to one of the apparatuses 200a, 200b, 200c, or 100. In accordance with various embodiments, the apparatus 200d can include any of the layers or any combination of the layers as shown to be included in the apparatuses 200a, 200b, 200c, or 100.

As shown in FIG. 2D, the apparatus 200d is depicted as having a channel 260d on one side and a compartment 280d on the other side. In accordance with various embodiments, the channel 260d can be the same or substantially similar to the channel 260 as described with respect to FIGS. 1C and 1D, unless stated otherwise, and therefore, will not be described in full detail. In accordance with various embodiments, the compartment 280d can be the same or substantially similar to the compartment 180 as described with respect to FIG. 1D, unless stated otherwise, and therefore, will not be described in full detail. As shown in FIG. 2D, the compartment 280d is formed in a material 205d that includes, for example, an electrically insulating material, including, but not limited to silicon nitride, silicon oxide, glass, a metal oxide, a carbide (such as, for example, SiCOH), a ceramic, such as, alumina, a polymer including plastic and various elastomers, such as poly(dimethyl siloxane) (PDMS), or any material that can be used as a structural material.

As shown in FIG. 2D, the apparatus includes an opening 225d. In accordance with various embodiments, the opening 225d can be the same or substantially similar to one of the openings 225a, 225b, and 225c. In accordance with various embodiments, the opening 225d can include a coating disposed thereon that is the same or substantially similar to the coating on the inner wall of the openings 225a, 225b, and 225c, unless stated otherwise, and therefore, will not be described in full detail.

As shown in FIG. 2D, the compartment 280d also includes an electrode layer 290d and a via 298d disposed in the electrode layer 290d, in accordance with various embodiments. In various implementations, the electrode layer 290d can be configured to actuate a sharp member, such as the sharp member 185 as described with respect to FIG. 1D. In accordance with various embodiments, the via 298d can be configured to pump a fluid in or out of the compartment 280d. In accordance with various embodiments, the fluid can include for example, but limited to, aqueous solution, aqueous solution containing biological or chemical reagents, organic solvents, mineral oil, fluorinated oil, air, mixed gases for cell culture (e.g. 5% CO2), inert gas, and the like.

In accordance with various embodiments, the apparatus 200d can include one or more coatings disposed on the surface and/or on the inside of the inner wall of the opening 225d. In accordance with various embodiments, the coatings on the surface and on the inside of the opening 225d can be the same or different. In accordance with various embodiments, the coating on the surface and the coating on the inside of the opening 225d can include the same pattern or different patterns.

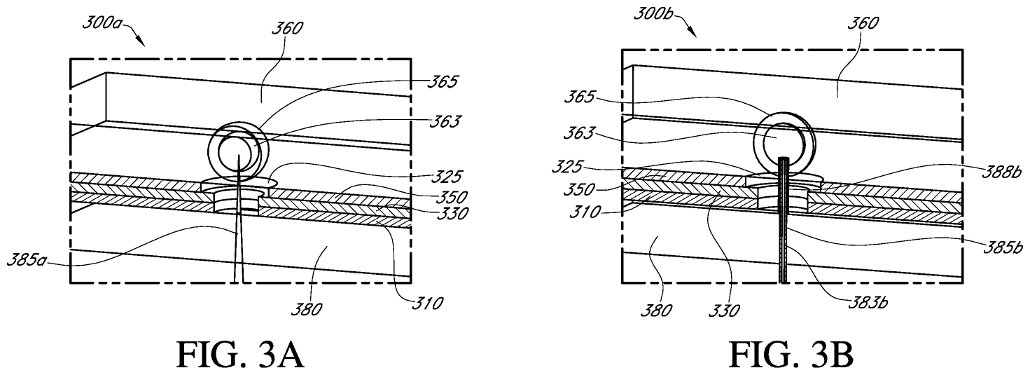

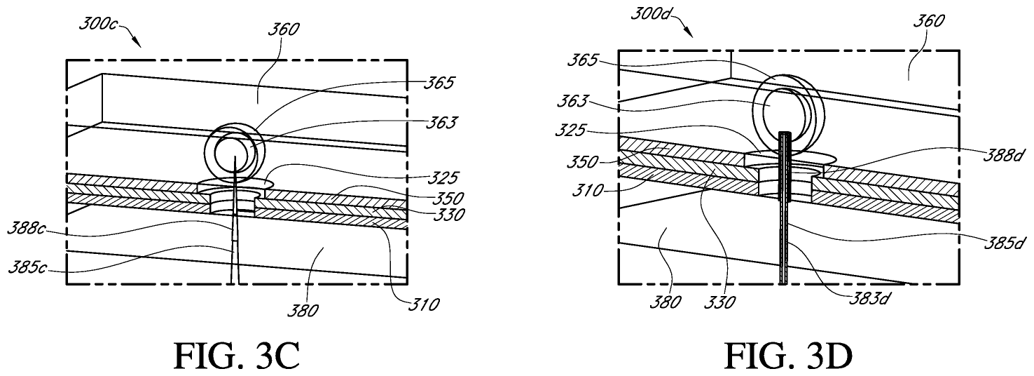

FIGS. 3A-3D show schematic illustrations 300a, 300b, 300c, and 300d, respectively, of an apparatus configured for interrogation of a particle, in accordance with various embodiments. The configurations shown in FIGS. 3A, 3B, 3C, and 3D are non-limiting examples, and thus, any desired structural configurations in addition to the illustrations can be utilized to perform immobilization and/or interrogation of a particle, in accordance with various embodiments.

As shown in FIGS. 3A-3D, the illustrations 300a, 300b, 300c, and 300d include a membrane 310, a metal layer 330, and a passivation layer 350. In accordance with various embodiments, the illustrations 300a, 300b, 300c, and 300d include an opening 325 across a channel 360 and a compartment 380. As shown in FIGS. 3A-3D, the illustrations 300a, 300b, 300c, and 300d also include a particle 365 having an inner portion 363 (e.g., nucleus or inner component) trapped, disposed, or otherwise immobilized approximate the opening 325. In accordance with various embodiments, the particle 365 is immobilized and ready for probing or interrogation.

In accordance with various embodiments, the membranes 310 can be the same or substantially similar to the membranes 110, 210a, 210b, or 210c as described with respect to FIGS. 1D, 2A, 2B, and 2C, unless stated otherwise, and therefore, will not be described in full detail.

In accordance with various embodiments, the metal layer 330 can be the same or substantially similar to the electrode(s) 120 and/or interconnect 130, or any of the metal layers 230a1, 230a2, 230b1, 230b2, 230c1, and 230c2 as described with respect to FIGS. 1A-1D, 2A-2C, unless stated otherwise, and therefore, will not be described in full detail.

In accordance with various embodiments, the passivation layer 350 can be the same or substantially similar to the passivation layers 150, 250a, 250b, or 250c as described with respect to FIGS. 1D, 2A, 2B, and 2C, unless stated otherwise, and therefore, will not be described in full detail.

In accordance with various embodiments, the opening 325 can be the same or substantially similar to one of the openings 125, 225a, 225b, 225c, or 225d as described with respect to FIGS. 1D, 2A, 2B, 2C, and 2D, unless stated otherwise, and therefore, will not be described in full detail.

In accordance with various embodiments, the opening 325 may include a coating disposed thereon that is the same or substantially similar to the coating on the inner wall of the openings 125, 225a, 225b, or 225c as described with respect to FIGS. 1D, 2A, 2B, and 2C, unless stated otherwise, and therefore, will not be described in full detail.

In accordance with various embodiments, the channel 360 can be the same or substantially similar to the channels 160 or 260d as described with respect to FIGS. 1C, 1D, and 2D, unless stated otherwise, and therefore, will not be described in full detail.

In accordance with various embodiments, the compartment 380 can be the same or substantially similar to the compartments 180 or 280d as described with respect to FIGS. 1D and 2D, unless stated otherwise, and therefore, will not be described in full detail.

As shown in FIGS. 3A-3D, each of the illustrations 300a, 300b, 300c, and 300d includes sharp members 385a, 385b, 385c, and 385d, respectively. FIG. 3A shows the sharp member 385a having a sharp tip. FIG. 3B shows the sharp member 385b having a hollow inner portion 383b and a coated tip 388b. FIG. 3C shows the sharp member 385c having a coating 388c disposed on its sharp tip. FIG. 3D shows the sharp member 385d having a hollow inner portion 383d and a coating 388d disposed on its tip.

As shown in FIGS. 3A-3D, the illustrations 300a, 300b, 300c, and 300d each show the respective sharp members 385a, 385b, 385c, and 385d (collectively referred to herein as “sharp members 385”) being inserted or probed (or interrogated) into the inner portion 363 of the particle 365. In accordance with various embodiments, each of the sharp members 385 is configured to move within the opening 325, and move through the membrane 310, the metal layer 330, and the passivation layer 350. According to various implementations, the opening 325 allows for mechanical manipulation of the particle 365 that is immobilized. In various implementations, the mechanical manipulation includes probing, inserting, penetrating, electroporating, sensing, depositing material, sampling material, or otherwise manipulating the particle 365 with the sharp members 385 configured to enter across the membrane 310, the metal layer 330, and/or the passivation layer 350. In various implementations, the mechanical manipulation is conducted by any of the sharp members 385. In various implementations, the sharp members 385 can be any of needle, a pillar, a hollow tube, nano-needle or a micro-needle having a length between about 10 nm to about 50 μm. In various implementations, the inner portions 383b and 383d can have an inner diameter from about 200 nm to about 100 μm, from about 10 nm to about 10 μm, or from about 1 nm to 1 μm. In various implementations, each of the sharp members 385 can be manufactured or fabricated via MEMS or NEMS methods, in accordance with various embodiments.

In various implementations, each of the sharp members 385 can be configured to operate as a third electrode in the form of a probe across the membrane 310. This third electrode probe may be biased with a DC or AC signal for sensing or actuation, for instance with a pulsed DC signal for Nanopore Electroporation (NEP) applications or with a low-power AC signal of a separate frequency for the purpose of measuring impedance. In various implementations, the DEP electrodes themselves may also carry a separate superposed AC or DC signal chosen to be easily isolated from the DEP signal via downstream filtering. In addition, a means of signal decoupling between the nanopore electroporation (NEP) signal and DEP signal may be implemented by physical shielding with materials, or careful signal control.

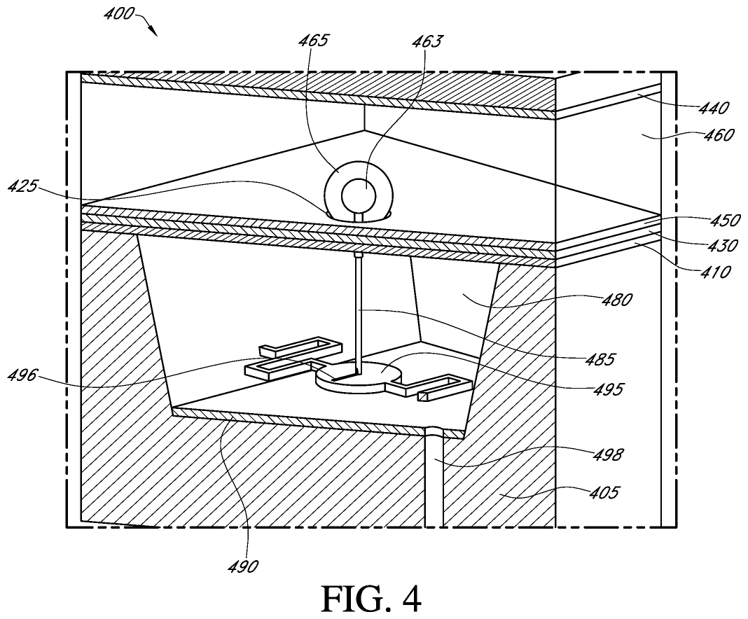

In various implementations, the sharp members 385 can enter from the compartment 380 where each of the sharp members 385 resides before its movement, e.g., before actuation of the sharp members 385 along the longitudinal axis, e.g., vertically upward. Additional details are provided with respect to FIG. 1D and further details will be provided with respect to FIG. 4.

FIG. 4 shows a schematic illustration of an apparatus 400 configured for location manipulation of a particle, in accordance with various embodiments. In accordance with various embodiments, the apparatus 400 can be the same or substantially similar to one of the apparatuses 100, 200a, 200b, 200c, or 200d as described with respect to FIGS. 1A-1D, 2A-2D. As shown in FIG. 4, the apparatus 400 includes a membrane 410, a metal layer 430, a passivation layer 150, and an opening 425. The illustration shown in FIG. 4 also includes a counter-electrode 440, a channel 460, and a compartment 480. As shown in FIG. 4, the illustration also includes a particle 465 having an inner portion 463 (e.g., nucleus or inner component) trapped, disposed, or otherwise immobilized approximate the opening 425.

In accordance with various embodiments, the channel 460 and the compartment 480 can each include a fluid. In accordance with various embodiments, the fluid includes one of an aqueous fluid, an aqueous buffer, an organic solvent, a hydrophobic fluid, or a gas. In accordance with various embodiments, the channel 460 can include a fluid (e.g., a first fluid) that is immiscible with a fluid (e.g., a second fluid) included in the compartment 480, or vice versa. For example, the fluid in the channel 460 can be a hydrophobic fluid while the fluid in the compartment 480 can be a hydrophilic fluid, or vice versa.

In accordance with various embodiments, the channel 460 can be configured to contain an aqueous solution, such as phosphate-buffered saline (PBS) or cell culture media, for the purpose of transporting cells or conducting biochemical reactions and the compartment 480 is configured to contain air or inert gas, in order to isolate sensitive electrical components from the aqueous solution of the channel 460.

In accordance with various embodiments, the channel 460 can be configured to contain an aqueous solution and the compartment 480 is configured to contain an organic solvent or oil, or vice versa, for a variety of purposes including for example, to protect electrical components that are sensitive to corrosion or electrolysis, for a chemical reaction that can use an organic solvent, or to sample small molecules.

In accordance with various embodiments, the channel 460 and the compartment 480 can be configured to contain dissimilar aqueous solutions in each chamber, for example, configuring the channel 460 to contain a solution carrying a suspension of cells and the compartment 480 to contain another solution with dissolved genetic material for delivering to captured cells via nanopore electroporation (NEP).

In accordance with various embodiments, the compartment 480 is formed in a material 405 that includes, for example, an electrically insulating material, including, but not limited to silicon nitride, silicon oxide, glass, a metal oxide, a carbide (such as, for example, SiCOH), a ceramic, such as, alumina, a polymer including plastic and various elastomers, such as poly(dimethyl siloxane) (PDMS), or any material that can be used as a structural material. As shown in FIG. 4, the compartment 480 also includes an electrode layer 490 and a via 498 disposed in the electrode layer 490, in accordance with various embodiments. In accordance with various embodiments, the via 498 can be configured to pump a fluid in or out of the compartment 480. In accordance with various embodiments, the fluid can include for example, but limited to, aqueous solution, aqueous solution containing biological or chemical reagents, organic solvents, mineral oil, fluorinated oil, air, mixed gases for cell culture (e.g. 5% CO2), inert gas, and the like.

As shown in FIG. 4, the compartment 480 also includes a substrate platform 495 on which a sharp member 485 is affixed. In accordance with various embodiments, the substrate platform 495 is configured to move against the electrode layer 490, via any suitable mechanism (e.g., via electrostatic force) as disclosed in various embodiments of this disclosure. For example, the substrate platform 495 can be configured to actuate to move up and down so as to move the sharp member 485, whereby the actuation enables the sharp member 485 to probe, insert, or interrogate the particle 465 and/or its inner portion 463.

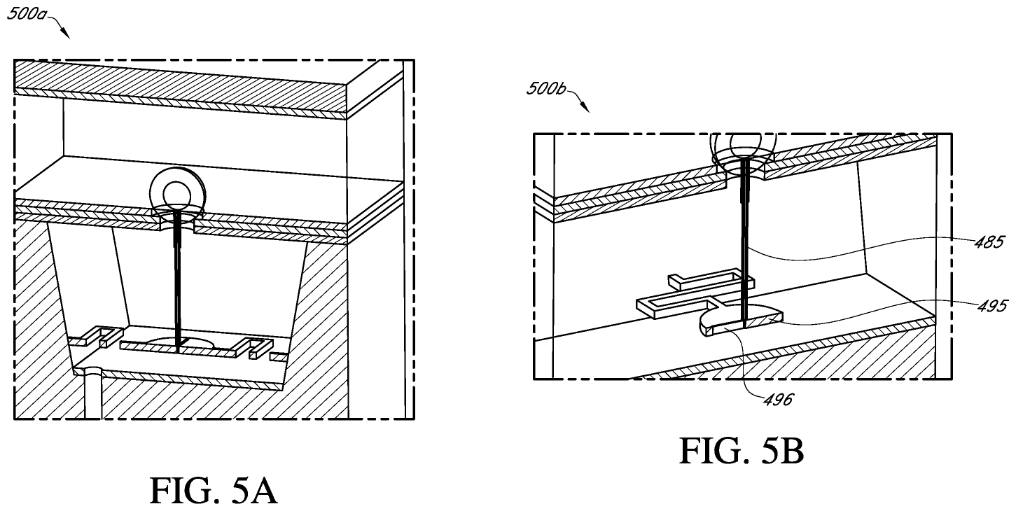

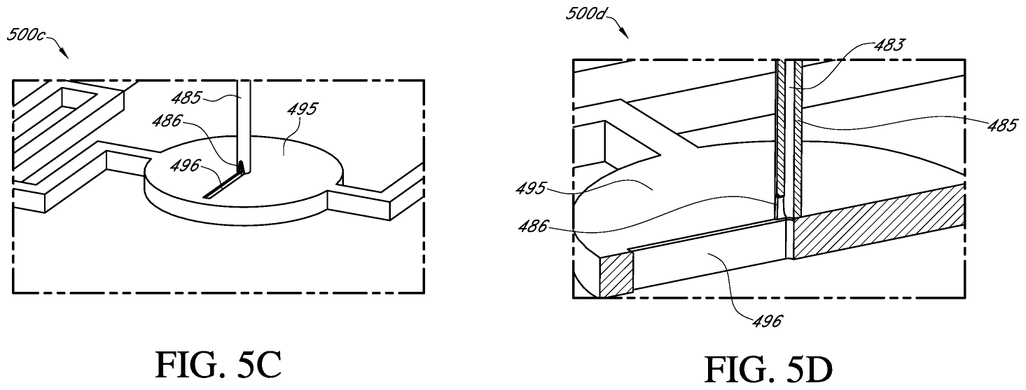

FIGS. 5A-5D are various schematic views of the apparatus 400 configured for location manipulation of a particle, in accordance with various embodiments. FIG. 5A shows a cross-sectional view of the apparatus 400 and FIG. 5B shows another view of the apparatus 400 to the view of FIG. 5A. FIGS. 5C and 5D show a zoomed-in perspective view and a zoomed-in cross-sectional view of the base of the sharp member 485 that is affixed to the substrate platform 495. As shown in FIGS. 5B, 5C, and 5D, the sharp member 485 is a hollow structure with an inner hollow (interior) portion 483. The illustrations of FIGS. 5C and 5D show a wicking structure 496 disposed within the substrate platform 495 and connected to an inlet 486 of the sharp member 485 to provide fluidic communication between the inner portion 483 and the interior of the compartment 480. In accordance with various embodiments, the combination of the wicking structure 496 and the inlet 486 is configured for a controlled fluidic communication that enables a controlled flow, such as, for example, but not limited to, electro-osmotic flow, electro-kinetic flow, capillary flow, or any other suitable flow or wicking mechanisms.

In various implementations, the wicking mechanisms can be used to supply any payload or payload mixture via the hollow portion 483 of the sharp member 485 and into the trapped or immobilized particle 465. In accordance with various embodiments, the hollow sharp member 485 can be configured to allow for particle penetration and electroporation via a fluid wicking path (e.g., a path through which the fluid is absorbed) from the substrate platform that resides within the compartment 480. In accordance with various embodiments, the compartment 480 can be filled with any suitable payload fluid, fluid mixture, or an inert non-polar liquid. In accordance with various embodiments, the payload may be delivered to any region of the particle 465, for example, to specific portion of a cell, such as a nucleus.

FIGS. 6A-6D illustrate various configurations of an apparatus configured for immobilization of a particle, in accordance with various embodiments. FIGS. 6A, 6B, and 6D illustrate non-limiting example electrode configurations for controlling an electric field across a given electrode pair. FIG. 6C illustrates a non-limiting example of an electrode configuration for controlling an electric field across an electrode pair and a ring counter-electrode.

FIG. 6A is an illustration of an electrode configuration 600a showing a top view of a pair of electrodes 620a that are disposed across an opening 625a. As shown in FIG. 6A, each of the electrodes 620a has a flat tip that generates straight electric field line between the two opposing flat tips from each of the electrodes 620a. The layout shown in FIG. 6A is constructed to trap or immobilize a particle approximate the opening 625a using the electric field lines generated across the two flat tips near the opening 625a. In accordance with various embodiments, the two tips of the electrodes 620 concentrate the electric field along the opening 625a. In accordance with various embodiments, surface areas outside of the electrodes 620a and the opening 625a are covered with a passivation material 650a, for example, to limit the stray electric field lines, to limit corrosion of electrodes or electrolysis, or to prevent electrical current flow in the bulk fluid.

FIG. 6B is an illustration of an electrode configuration 600b showing a top view of a pair of electrodes 620b that are disposed across an opening 625b. As shown in FIG. 6B, each of the electrodes 620b has a sharp tip that generates focused electric field lines between the two opposing sharp tips from each of the electrodes 620b. The layout shown in FIG. 6B is constructed to trap or immobilize a particle approximate the opening 625b using a more focused electric field generated across the two sharp tips near the opening 625b. In accordance with various embodiments, the electric field lines generated between the two sharp tips of the electrodes 620b are nonlinear and focused at the sharp tips. In accordance with various embodiments, surface areas outside of the electrodes 620b and the opening 625b are covered with a passivation material 650b, for example, to limit the stray electric field lines, to limit corrosion of electrodes or electrolysis, or to prevent electrical current flow in the bulk fluid.

FIG. 6C is an illustration of an electrode configuration 600c showing a pair of electrodes similar to those shown in FIG. 6A, as well as a ring electrode 622c. As shown in FIG. 6A, each of the electrodes 620c is connected to buried interconnects 630c, which is separated from the ring electrode 622c by a layer of dielectric material 650c, similar to the configuration shown in FIG. 7C. In accordance with various embodiments, the pair of electrodes 620c are configured to function similarly to the electrodes 620a and 620b, shown in FIGS. 6A and 6B, i.e., to generate a concentrated electric field localized around an opening 625c. In accordance with various embodiments, the ring electrode 622c is configured as a common ground for the two electrodes 620c, confining the in-plane stray electric field to the area around the trapping site, i.e., the opening 625c. In accordance with various embodiments, surface areas outside of the electrodes 620c, ring electrode 622c, and the opening 625c are covered with a passivation material 650c, for example, to limit the stray electric field lines, to limit corrosion of electrodes or electrolysis, or to prevent electrical current flow in the bulk fluid.

FIG. 6D is an illustration of an electrode configuration showing a cross-sectional view of an example electrode configuration 600d, in accordance with various embodiments. As shown in FIG. 6D, the electrode configuration 600d includes a pair of electrodes 620d and a passivation (dielectric) material 650d that are disposed on a membrane 610d and across an opening 625d.

FIGS. 7A-7C show schematic illustrations of various example configurations of an apparatus configured for immobilization of a plurality of particles, in accordance with various embodiments. As shown in FIGS. 7A-7C, the apparatus includes an insulation layer 750, electrodes 720, interconnects 730, and a dielectric layer 752 that are stacked on top of each other and are disposed on a membrane 710. In accordance with various embodiments, the insulation layer 750 includes a window 704 in the insulation layer 750 that exposes a top surface portion of each of the electrodes 720.

FIG. 7A shows a perspective view of an example electrode configuration 700a of an apparatus having an array of electrodes for immobilizing and/or interrogation, in accordance with various embodiments. As shown in FIG. 7A, the configuration 700a includes a plurality of pairs of electrodes 720 that are disposed across each of a plurality of openings 725. The configuration 700a also includes a plurality of interconnects 730 that are configured for interconnecting various electrodes 720. In accordance with various embodiments, the interconnects 730 are disposed in the same layer as the electrodes 720.

FIG. 7B shows a perspective view of another example electrode configuration 700b of an apparatus having an array of electrodes for immobilizing and/or interrogation, in accordance with various embodiments. As shown in FIG. 7B, the configuration 700b includes a plurality of pairs of electrodes 720 that are disposed across each of a plurality of openings 725. The configuration 700a also includes a plurality of interconnects 730 that are configured for interconnecting various electrodes 720. In accordance with various embodiments, the interconnects 730 are disposed on a different layer as the electrodes 720, as shown in FIG. 7B.

FIG. 7C shows a cross-sectional view 700c of the electrode configuration 700b. As shown in FIG. 7C, the cross-section along the lines A-A′ of the apparatus shows how the electrodes 720 are interfaced with the interconnects 730, which are disposed within the dielectric layer 752. The dielectric layer 752 is disposed below the electrodes 720. In accordance with various embodiments, the interconnects 730 is embedded in the dielectric layer 752 and interfaced with the electrodes 720 vertically.

In various implementations, each electrode in the pair of electrodes 620a, 620b, 620c, 620d, and 720 can be operated out of phase at a phase shift of about 180 degrees with respect to the other electrode in each pair of electrodes. In various implementations, the phase shift can be 360 degrees/number of electrodes, for example, a phase shift of 120 degrees if it is a three-electrode configuration, or a phase shift of 90 degrees for a 4-electrode configuration, that is being used for trapping or immobilizing.

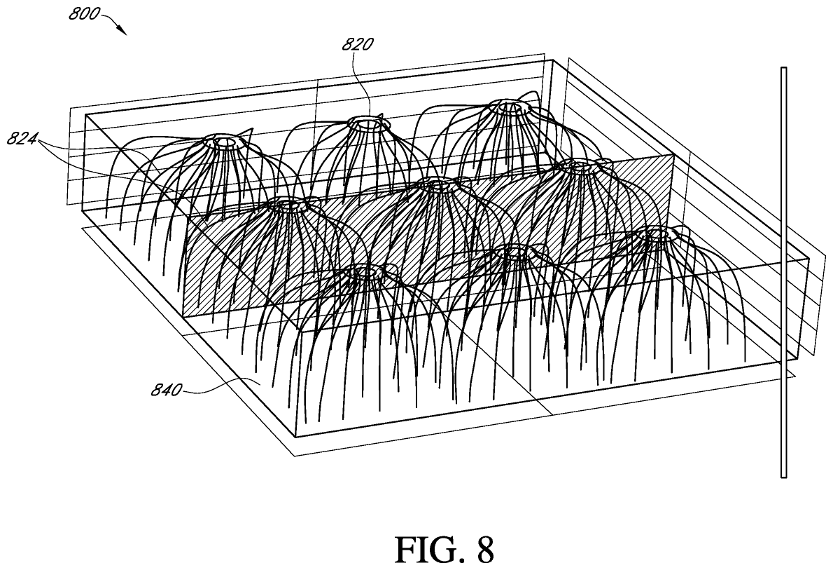

FIG. 8 is a graphical diagram 800 displaying simulation results for an apparatus for immobilization of a particle (not shown). As shown in FIG. 8, an AC field is supplied across a plurality of electrodes 820 and a counter-electrode 840. A DEP force on the order of tens to hundreds of nanonewtons (nN) is generated across the plurality of electrodes 820 and the counter-electrode 840. The generated DEP can trap or immobilize a particle (or a cell), for example, against a fluid velocity up to centimeters per second (cm/s). The simulation in the graphical diagram 800 shown in FIG. 8 is generated using a simulation software program, to illustrate electric field lines 824 with a maximum field of 70 kV/m in a simulated 5 V oscillating at 1 MHz across the plurality of electrodes 820 and the counter-electrode 840.

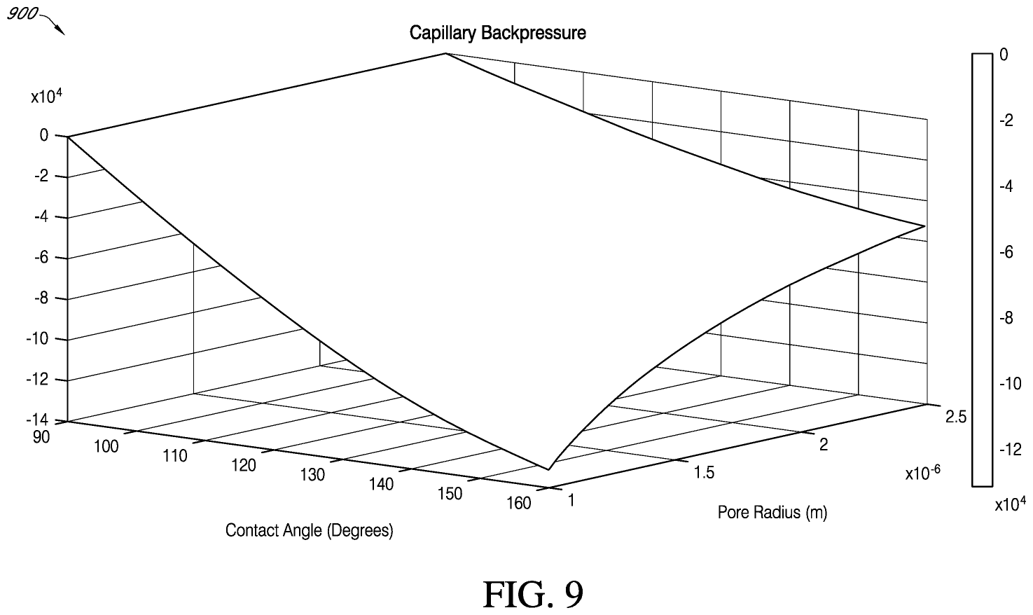

FIG. 9 is a three-dimensional chart 900 showing results of an analysis for an apparatus for immobilization of a particle. As shown in FIG. 9, the capillary backpressure (in Pascal) as a function of contact angle and a radius of an opening is calculated from the capillary pressure equation described above in the case of a water-air interface. For example, negative values shown in the chart 900 correspond to pressure in the direction of the fluid, e.g., away from the compartment, for example, that houses MEMS components.

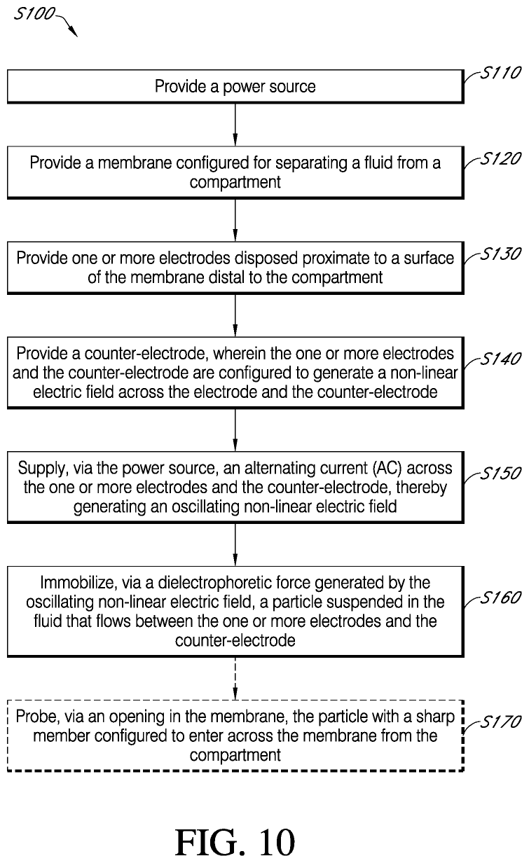

FIG. 10 is a flow chart for an example method S100 of operating an apparatus for immobilization of a particle, according to an illustrative implementation. As shown in FIG. 10, the method S100 includes providing a power source at step S110. The method S100 also includes providing a membrane configured for separating a fluid from a compartment at step S120. The method S100 also includes providing one or more electrodes disposed proximate to the membrane at step S130. In accordance with various embodiments, the one or more electrodes is disposed proximate to a surface of the membrane, the surface distal to the compartment. In accordance with various embodiments, the one or more electrodes is disposed proximate to a surface of the membrane, the surface proximate to the compartment. The method S100 also includes providing a counter-electrode, wherein the one or more electrodes and the counter-electrode are configured to generate a non-linear electric field across the one or more electrodes and the counter-electrode at step S140.

As shown in FIG. 10, the method S100 includes supplying, via the power source, an alternating current (AC) across the one or more electrodes and the counter-electrode, thereby generating an oscillating non-linear electric field at step S150. The method S100 also includes immobilizing, via a dielectrophoretic (DEP) force generated by the oscillating non-linear electric field, a particle suspended in the fluid that flows between the one or more electrodes and the counter-electrode at step S160. The method S100 optionally includes probing, via an opening in the membrane, the particle with a sharp member configured to enter across the membrane from the compartment at step S170. In various implementations, the sharp member includes a MEMS structure or a NEMS structure.

In various implementations, the method optionally includes manipulating, via the opening, the particle that is immobilized. In various implementations, the method optionally includes inserting, via the opening, the particle with a sharp member configured to enter across the membrane from the compartment.

In various implementations of the method S100, the membrane includes at least one of silicon nitride, silicon oxide, a metal oxide, a carbide, a ceramic, alumina, or a polymer. In various implementations of the method S100, the membrane has a thickness between about 10 nm to about 1 cm. In various implementations, the membrane has a thickness between about 100 nm to about 10 μm. In various implementations of the method S100, the opening has a size between about 10 nm to about 50 μm. In various implementations, the opening has a size between about 1 μm to about 5 μm.

In various implementations of the method S100, a wall of the opening has a hydrophobic coating or a hydrophilic coating. In various implementations of the method S100, the hydrophobic coating has a contact angle between about 95° and about 165°. In various implementations of the method S100, the hydrophilic coating has a contact angle between about 20° and about 80°.

In various implementations of the method S100, the first surface is smaller than the second surface. In various implementations of the method S100, the one or more electrodes includes a plurality of individual disparate one or more electrodes surface areas formed in an array.

In various implementations of the method S100, the AC across the one or more electrodes and the counter-electrode is supplied at a voltage between about 1 mV and about 300 V. In various implementations, the AC across the one or more electrodes and the counter-electrode is supplied at a voltage between about 1 mV and about 20 V.

In various implementations of the method S100, the AC across the one or more electrodes and the counter-electrode is supplied at an oscillating frequency of between about 10 Hz and about 10 GHz. In various implementations, the AC across the one or more electrodes and the counter-electrode is supplied at an oscillating frequency of between about 1 kHz and about 1 GHz.

In various implementations of the method S100, the one or more electrodes includes at least one of a transparent conducting material or a doped semiconducting material. In various implementations, the transparent conducting material includes indium tin oxide, graphene, doped graphene, a conducting polymer, or a thin metal layer.

In various implementations of the method S100, the one or more electrodes has a thickness between about 1 nm to about 50 μm. In various implementations, the one or more electrodes has a thickness between about 10 nm to about 5 μm.

In various implementations of the method S100, the fluid includes one of an aqueous fluid, an aqueous buffer, an organic solvent, a hydrophobic fluid, or a gas. In various implementations of the method S100, the fluid is a first fluid, the compartment comprises a second fluid that is immiscible with the first fluid. In various implementations of the method S100, the first fluid is a hydrophobic fluid and the second fluid is a hydrophilic fluid, or vice versa.

In various implementations of the method S100, the particle has a size between about 1 nm to about 1 mm. In various implementations, the particle includes one of a biological organism, a biological structure, a cell, a living cell, viruses, oil droplets, liposomes, micelles, reverse micelles, protein aggregates, polymers, or surfactant assemblies.

In various implementations, for individual addressing of the site where the particle is immobilized for probing (can refer to as “probing site”), a feedback control mechanism can be configured via impedance sensing to allow for optimization of cell capture in an automated workflow. In various implementations, a particle trapping event can be detected via impedance sensing using a superposed sensing frequency that is filtered by capacitance measurement of the particle (e.g., by measuring capacitance of the membrane of a cell). This frequency can then be isolated from the driving dielectrophoretic (DEP) frequency by a filter circuit and the magnitude and phase information at this frequency is correlated with the expected effect of a trapped particle.

In various implementations, should an unexpected signal be detected, possibly indicating the trapping of more than one particle, for example, an undesired particle or cell type, or a piece of dust, etc., the DEP electrodes can be turned off, thereby allowing the flow to dispose of the particle. Then the trapping procedure can be reattempted. In various implementations, a real-time optimization can be carried out by recirculating flow until a sufficient percentage of particles (or cells) are captured at the probing sites, adjusting the signal voltage and flow rate accordingly. In various implementations, the procedure is similar, but a third electrode is present in a MEMS probe that is inserted into the cell interior through the pore, allowing for a direct impedance measurement from the interior of the cell.

In various implementations, the compartment (e.g., the cavity region) is conductive to allow for an electrical signal to be applied to the fluid contents contained within. This cavity is separated by a membrane from the fluid flowing region with a pore or plurality of pores and accompanying DEP electrodes spatially overlaid with each pore in the same fashion as previous embodiments. Particles of any type including living cells, and/or vesicles may be trapped and electroporated via the signal applied to the fluid contents of the cavity transmitted through the membrane pore in order to allow the addressed electroporation of cell arrays. This embodiment also can include a counter-electrode on the top of the fluid flow region. In addition, a means of signal decoupling between the nanopore electroporation (NEP) signal and DEP signal may be implemented by physical shielding with materials, or careful signal control.

Similarly, in various implementations, the NEP cavity (previously the MEMS cavity) can be configured with a fluid input channel that can supply any payload or payload mixture to the cavity for subsequent NEP delivery into a DEP trapped particle. These fluid input channels may be multiplexed (e.g., combined, redirected, etc.) in an array coming from multiple sources with different payload compositions or may be configured to supply one type of payload composition. A single array of these NEP-DEP (probing) sites can be sectored on a chip to include sectors with a multiplexed configuration and/or a single source configuration on one chip.

In various implementations, the hollow probe (e.g., a sharp member) is configured to allow for particle penetration and electroporation via a signal applied to the probe. A fluid wicking path (e.g., a path through which the fluid is absorbed) from the MEMS stage allows for payload transit from the MEMS cavity through the hollow probe up to the particle. In one such implementation, the MEMS cavity is filled with a uniform payload fluid mixture. In another such implementation, the MEMS cavity is filled with an inert non-polar liquid and the fluid wicking path up through the inside of the hollow probe to its tip is filled with a polar liquid and payload mixture. During operation this hollow probe may be actuated and inserted at any depth within the DEP trapped particle after which a signal is applied to the probe for electroporation and payload delivery. In this manner the payload may be delivered to any region of the particle and in the cases of a cell or the nucleus.

In various implementations, the hollow probe may be configured to receive a signal that allows for the variable sorption or desorption of payload solution within its interior with high volumetric precision in order to allow for physical volumetric injection or sampling of fluid at different regions within a particle, cell, or vesicle for example.

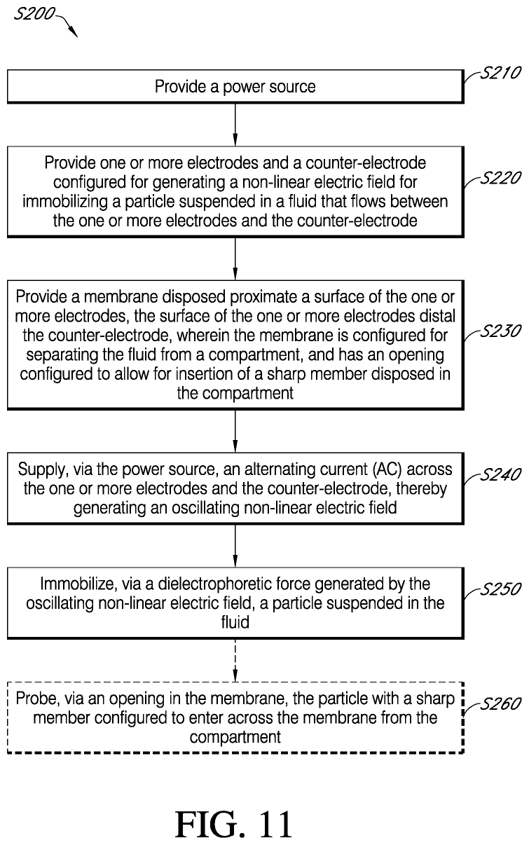

FIG. 11 is a flow chart for an example method S200 of operating an apparatus for immobilization of a particle, according to an illustrative implementation. As shown in FIG. 11, the method S200 includes providing a power source at step S210. The method S200 also includes providing one or more electrodes and a counter-electrode configured for generating a non-linear electric field for immobilizing a particle suspended in a fluid that flows between the one or more electrodes and the counter-electrode at step S220. The method S200 also includes providing a membrane disposed proximate a surface of the one or more electrodes, the surface of the one or more electrodes distal the counter-electrode, wherein the membrane is configured for separating the fluid from a compartment, and has an opening configured to allow for insertion of a sharp member disposed in the compartment at step S230.

As shown in FIG. 11, the method S200 also includes supplying, via the power source, an alternating current (AC) across the one or more electrodes and the counter-electrode, thereby generating an oscillating non-linear electric field at step S240. The method S200 also includes immobilizing, via a dielectrophoretic force generated by the oscillating non-linear electric field, a particle suspended in the first fluid at step S250. The method S200 optionally includes probing, via an opening in the membrane, the particle with a sharp member configured to enter across the membrane from the compartment at step S260. In various implementations, the sharp member includes a MEMS structure or a NEMS structure.

In various implementations, the method optionally includes manipulating, via the opening, the particle that is immobilized. In various implementations, the method optionally includes inserting, via the opening, the particle with a sharp member configured to enter across the membrane from the compartment.

In various implementations of the method S200, the membrane includes at least one of silicon nitride, silicon oxide, a metal oxide, a carbide, a ceramic, alumina, or a polymer. In various implementations of the method S200, the membrane has a thickness between about 10 nm to about 1 cm. In various implementations, the membrane has a thickness between about 100 nm to about 10 μm. In various implementations of the method S200, the opening has a size between about 10 nm to about 50 μm. In various implementations, the opening has a size between about 1 μm to about 5 μm.

In various implementations of the method S200, a wall of the opening has a hydrophobic coating or a hydrophilic coating. In various implementations, the hydrophobic coating has a contact angle between about 95° and about 165°. In various implementations of the method S100, the hydrophilic coating has a contact angle between about 20° and about 80°.

In various implementations, the first surface is smaller than the second surface. In various implementations of the method S200, the one or more electrodes includes a plurality of individual disparate electrode surface areas formed in an array.

In various implementations, the AC across the one or more electrodes and the counter-electrode is supplied at a voltage between about 1 mV and about 300 V. In various implementations, the AC across the one or more electrodes and the counter-electrode is supplied at a voltage between about 1 mV and about 20 V.

In various implementations, the AC across the one or more electrodes and the counter-electrode is supplied at an oscillating frequency of between about 10 Hz and about 10 GHz. In various implementations, the AC across the one or more electrodes and the counter-electrode is supplied at an oscillating frequency of between about 1 kHz and about 1 GHz.

In various implementations, the one or more electrodes includes at least one of a transparent conducting material or a doped semiconducting material. In various implementations, the transparent conducting material includes indium tin oxide, graphene, doped graphene, a conducting polymer, or a thin metal layer.

In various implementations of the method S200, the one or more electrodes has a thickness between about 1 nm to about 50 μm. In various implementations, the one or more electrodes has a thickness between about 10 nm to about 5 μm.

In various implementations of the method S200, the fluid includes one of an aqueous fluid, an aqueous buffer, an organic solvent, a hydrophobic fluid, or a gas. In various implementations of the method S200, the fluid is a first fluid, the compartment comprises a second fluid that is immiscible with the first fluid. In various implementations of the method S200, the first fluid is a hydrophobic fluid and the second fluid is a hydrophilic fluid, or vice versa.

In various implementations, the particle has a size between about 1 nm to about 1 mm. In various implementations, the particle includes one of a biological organism, a biological structure, a cell, a living cell, viruses, oil droplets, liposomes, micelles, reverse micelles, protein aggregates, polymers, or surfactant assemblies.

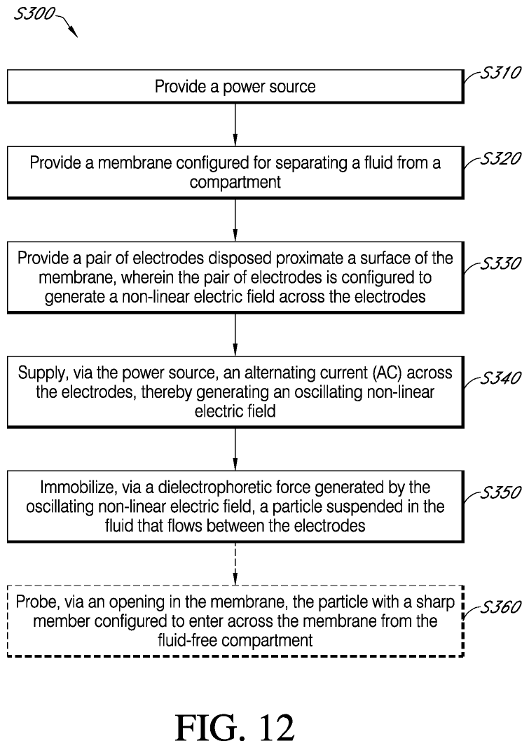

FIG. 12 is a flow chart for an example method S300 of operating an apparatus for immobilization of a particle, in accordance with various embodiments. As shown in FIG. 12, the method S300 includes providing a power source at step S310. The method S300 also includes providing a membrane configured for separating a fluid from a compartment at step S320. The method S300 also includes providing a pair of electrodes disposed proximate a surface of the membrane, wherein the pair of electrodes is configured to generate a non-linear electric field across the electrodes at step S330.

As shown in FIG. 12, the method S300 also includes supplying, via the power source, an alternating current (AC) across the electrodes, thereby generating an oscillating non-linear electric field at step S340. The method S300 also includes immobilizing, via a dielectrophoretic force generated by the oscillating non-linear electric field, a particle suspended in the fluid that flows between the electrodes at step S350. The method S300 optionally includes probing, via an opening in the membrane, the particle with a sharp member configured to enter across the membrane from the compartment at step S360. In various implementations, the sharp member includes a MEMS structure or a NEMS structure.

In various implementations, the method optionally includes providing a counter-electrode. In various implementations, the method optionally includes providing a third electrode disposed proximate the surface of the membrane. In various implementations, the third electrode is a ring electrode. In various implementations, the method optionally includes manipulating, via the opening, the particle that is immobilized. In various implementations, the method optionally includes inserting, via the opening, the particle with a sharp member configured to enter across the membrane from the compartment.

In various implementations, each of the pair of electrodes comprises a sharp tip or flat tip. In various implementations, the membrane comprises at least one of silicon nitride, silicon oxide, a metal oxide, a carbide, a ceramic, alumina, or a polymer. In various implementations, the membrane has a thickness between about 10 nm to about 1 cm. In various implementations, the membrane has a thickness between about 100 nm to about 10 μm.

In various implementations, the opening has a size between about 10 nm to about 50 μm. In various implementations, the opening has a size between about 1 μm to about 5 μm.

In various implementations, a wall of the opening has a hydrophobic coating or a hydrophilic coating. In various implementations, the hydrophobic coating has a contact angle between about 95° and about 165°. In various implementations, the hydrophilic coating has a contact angle between about 20° and about 80°.

In various implementations, the first surface is smaller than the second surface. In various implementations, the membrane includes a plurality of electrode pairs formed in an array.

In various implementations, the AC across the pair of electrodes and the counter-electrode is supplied at a voltage between about 1 mV and about 300 V. In various implementations, the AC across the pair of electrodes and the counter-electrode is supplied at a voltage between about 1 mV and about 20 V.

In various implementations, the AC across the pair of electrodes and the counter-electrode is supplied at an oscillating frequency of between about 10 Hz and about 10 GHz. In various implementations, the AC across the pair of electrodes and the counter-electrode is supplied at an oscillating frequency of between about 1 kHz and about 1 GHz.

In various implementations, one electrode of the pair of electrodes includes at least one of a transparent conducting material or a doped semiconducting material. In various implementations, the transparent conducting material includes indium tin oxide, graphene, doped graphene, a conducting polymer, or a thin metal layer.

In various implementations, the pair of electrodes has a thickness between about 1 nm to about 50 μm. In various implementations, the pair of electrodes has a thickness between about 10 nm to about 5 μm.

In various implementations, the fluid includes one of an aqueous fluid, an aqueous buffer, an organic solvent, a hydrophobic fluid, or a gas. In various implementations of the method S300, the fluid is a first fluid, the compartment comprises a second fluid that is immiscible with the first fluid. In various implementations of the method S300, the first fluid is a hydrophobic fluid and the second fluid is a hydrophilic fluid, or vice versa.

### Recitation of Embodiments

EMBODIMENT 1. An apparatus comprising a membrane for separating a fluid from a compartment; one or more electrodes disposed proximate to the membrane; a counter-electrode, wherein the one or more electrodes and the counter-electrode are configured to generate a non-linear electric field across the one or more electrodes and the counter-electrode; and a power source for providing an alternating current (AC) across the one or more electrodes and the counter-electrode, thereby generating an oscillating non-linear electric field for immobilizing a particle suspended in the fluid that flows between the one or more electrodes and the counter-electrode.

EMBODIMENT 2. The apparatus of embodiment 1, wherein the membrane comprises an opening.

EMBODIMENT 3. The apparatus of embodiment 2, wherein the opening allows for mechanical manipulation of the particle that is immobilized and the mechanical manipulation includes probing the particle with a sharp member configured to enter across the membrane from the compartment.

EMBODIMENT 4. The apparatus of any preceding embodiment, wherein the membrane comprises at least one of silicon nitride, silicon oxide, a metal oxide, a carbide, a ceramic, alumina, or a polymer.

EMBODIMENT 5. The apparatus of any preceding embodiment, wherein the membrane has a thickness between about 10 nm to about 1 cm.

EMBODIMENT 6. The apparatus of any preceding embodiment, wherein the membrane has a thickness between about 100 nm to about 10 μm.

EMBODIMENT 7. The apparatus of embodiment 2, wherein the opening has a size between about 10 nm to about 50 μm.

EMBODIMENT 8. The apparatus of any preceding embodiment, wherein the opening has a size between about 1 μm to about 5 μm.

EMBODIMENT 9. The apparatus of any preceding embodiment, wherein a wall of the opening has a hydrophobic coating or a hydrophilic coating.

EMBODIMENT 10. The apparatus of embodiment 9, wherein the hydrophobic coating has a contact angle between about 95° and about 165°.

EMBODIMENT 11. The apparatus of any preceding embodiment, wherein the hydrophilic coating has a contact angle between about 20° and about 80°.

EMBODIMENT 12. The apparatus of any preceding embodiment, wherein a surface area of the one or more electrodes is smaller than a surface area of the counter-electrode.

EMBODIMENT 13. The apparatus of any preceding embodiment, wherein the one or more electrodes comprises a plurality of individual disparate electrode surface areas formed in an array.

EMBODIMENT 14. The apparatus of any preceding embodiment, wherein the AC across the one or more electrodes and the counter-electrode is supplied at a voltage between about 1 mV and about 300 V.

EMBODIMENT 15. The apparatus of any preceding embodiment, wherein the AC across the one or more electrodes and the counter-electrode is supplied at a voltage between about 1 mV and about 20 V.

EMBODIMENT 16. The apparatus of any preceding embodiment, wherein the AC across the one or more electrodes and the counter-electrode is supplied at an oscillating frequency of between about 10 Hz and about 10 GHz.

EMBODIMENT 17. The apparatus of any preceding embodiment, wherein the AC across the one or more electrodes and the counter-electrode is supplied at an oscillating frequency of between about 1 kHz and about 1 GHz.

EMBODIMENT 18. The apparatus of any preceding embodiment, wherein the one or more electrodes comprises at least one of a transparent conducting material or a doped semiconducting material.

EMBODIMENT 19. The apparatus of embodiment 18, wherein the transparent conducting material comprises indium tin oxide, graphene, doped graphene, a conducting polymer, or a thin metal layer.

EMBODIMENT 20. The apparatus of any preceding embodiment, wherein the one or more electrodes has a thickness between about 1 nm to about 50 μm.

EMBODIMENT 21. The apparatus of any preceding embodiment, wherein the one or more electrodes has a thickness between about 10 nm to about 5 μm.

EMBODIMENT 22. The apparatus of any preceding embodiment, wherein the fluid comprises one of an aqueous fluid, an aqueous buffer, an organic solvent, a hydrophobic fluid, or a gas.

EMBODIMENT 23. The apparatus of any preceding embodiment, wherein the particle has a size between about 1 nm to about 1 mm.

EMBODIMENT 24. The apparatus of any preceding embodiment, wherein the particle comprises one of a biological organism, a biological structure, a cell, a living cell, viruses, oil droplets, liposomes, micelles, reverse micelles, protein aggregates, polymers, or surfactant assemblies.

EMBODIMENT 25. The apparatus of any preceding embodiment, wherein the compartment comprises a Micro-Electro-Mechanical System (MEMS) structure or a Nano-Electro-Mechanical System (NEMS) structure.

EMBODIMENT 26. A method for operating an apparatus comprising providing a power source; providing a membrane configured for separating a fluid from a compartment; providing one or more electrodes disposed proximate to the membrane; providing a counter-electrode, wherein the one or more electrodes and the counter-electrode are configured to generate a non-linear electric field across the one or more electrodes and the counter-electrode; supplying, via the power source, an alternating current (AC) across the one or more electrodes and the counter-electrode, thereby generating an oscillating non-linear electric field; and immobilizing, via a dielectrophoretic force generated by the oscillating non-linear electric field, a particle suspended in the fluid that flows between the one or more electrodes and the counter-electrode.

EMBODIMENT 27. The method of embodiment 26, wherein the membrane comprises an opening.

EMBODIMENT 28. The method of any preceding embodiment, further comprising manipulating, via the opening, the particle that is immobilized.

EMBODIMENT 29. The method of any preceding embodiment, further comprising probing, via the opening, the particle with a sharp member configured to enter across the membrane from the compartment.

EMBODIMENT 30. The method of any preceding embodiment, further comprising inserting, via the opening, the particle with a sharp member configured to enter across the membrane from the compartment.

EMBODIMENT 31. The method of embodiment 30, wherein the sharp member comprises a Micro-Electro-Mechanical System (MEMS) structure or a Nano-Electro-Mechanical System (NEMS) structure.

EMBODIMENT 32. The method of any preceding embodiment, wherein the membrane comprises at least one of silicon nitride, silicon oxide, a metal oxide, a carbide, a ceramic, alumina, or a polymer.

EMBODIMENT 33. The method of any preceding embodiment, wherein the membrane has a thickness between about 10 nm to about 1 cm.

EMBODIMENT 34. The method of any preceding embodiment, wherein the membrane has a thickness between about 100 nm to about 10 μm.

EMBODIMENT 35. The method of any preceding embodiment, wherein the opening has a size between about 10 nm to about 50 μm.

EMBODIMENT 36. The method of any preceding embodiment, wherein the opening has a size between about 1 μm to about 5 μm.

EMBODIMENT 37. The method of any preceding embodiment, wherein a wall of the opening has a hydrophobic coating or a hydrophilic coating.

EMBODIMENT 38. The method of embodiment 37, wherein the hydrophobic coating has a contact angle between about 95° and about 165°.

EMBODIMENT 39. The method of any preceding embodiment, wherein the hydrophilic coating has a contact angle between about 20° and about 80°.

EMBODIMENT 40. The method of any preceding embodiment, wherein a surface area of the one or more electrodes is smaller than a surface area of the counter-electrode.

EMBODIMENT 41. The method of any preceding embodiment, wherein the one or more electrodes comprises a plurality of individual disparate electrode surface areas formed in an array.

EMBODIMENT 42. The method of any preceding embodiment, wherein the AC across the one or more electrodes and the counter-electrode is supplied at a voltage between about 1 mV and about 300 V.

EMBODIMENT 43. The method of any preceding embodiment, wherein the AC across the one or more electrodes and the counter-electrode is supplied at a voltage between about 1 mV and about 20 V.

EMBODIMENT 44. The method of any preceding embodiment, wherein the AC across the one or more electrodes and the counter-electrode is supplied at an oscillating frequency of between about 10 Hz and about 10 GHz.

EMBODIMENT 45. The method of any preceding embodiment, wherein the AC across the one or more electrodes and the counter-electrode is supplied at an oscillating frequency of between about 1 kHz and about 1 GHz.

EMBODIMENT 46. The method of any preceding embodiment, wherein the one or more electrodes comprises at least one of a transparent conducting material or a doped semiconducting material.

EMBODIMENT 47. The method of any preceding embodiment, wherein the transparent conducting material comprises indium tin oxide, graphene, doped graphene, a conducting polymer, or a thin metal layer.

EMBODIMENT 48. The method of any preceding embodiment, wherein the one or more electrodes has a thickness between about 1 nm to about 50 μm.

EMBODIMENT 49. The method of any preceding embodiment, wherein the one or more electrodes has a thickness between about 10 nm to about 5 μm.

EMBODIMENT 50. The method of any preceding embodiment, wherein the fluid comprises one of an aqueous fluid, an aqueous buffer, an organic solvent, a hydrophobic fluid or a gas.

EMBODIMENT 51. The method of any preceding embodiment, wherein the particle has a size between about 1 nm to about 1 mm.

EMBODIMENT 52. The method of any preceding embodiment, wherein the particle comprises one of a biological organism, a biological structure, a cell, a living cell, viruses, oil droplets, liposomes, micelles, reverse micelles, protein aggregates, polymers, or surfactant assemblies.

EMBODIMENT 53. An apparatus comprising one or more electrodes and a counter-electrode configured for generating a non-linear electric field for immobilizing a particle suspended in a fluid that flows between the one or more electrodes and the counter-electrode; and a membrane disposed proximate a surface of the one or more electrodes, the surface of the one or more electrodes distal the counter-electrode, wherein the membrane is configured for separating the fluid from a compartment, and has an opening configured to allow for insertion of a sharp member disposed in the compartment.

EMBODIMENT 54. The apparatus of embodiment 53, wherein the sharp member is a Micro-Electro-Mechanical System (MEMS) structure or a Nano-Electro-Mechanical System (NEMS) structure.

EMBODIMENT 55. The apparatus of any preceding embodiment, wherein the membrane comprises at least one of silicon nitride, silicon oxide, a metal oxide, a carbide, a ceramic, alumina, or a polymer.

EMBODIMENT 56. The apparatus of any preceding embodiment, wherein the membrane has a thickness between about 10 nm to about 1 cm.

EMBODIMENT 57. The apparatus of any preceding embodiment, wherein the membrane has a thickness between about 100 nm to about 10 μm.

EMBODIMENT 58. The apparatus of any preceding embodiment, wherein the opening has a size between about 10 nm to about 50 μm.

EMBODIMENT 59. The apparatus of any preceding embodiment, wherein the opening has a size between about 1 μm to about 5 μm.

EMBODIMENT 60. The apparatus of any preceding embodiment, wherein a wall of the opening has a hydrophobic coating or a hydrophilic coating.

EMBODIMENT 61. The apparatus of embodiment 60, wherein the hydrophobic coating has a contact angle between about 95° and about 165°.

EMBODIMENT 62. The apparatus of any preceding embodiment, wherein the hydrophilic coating has a contact angle between about 20° and about 80°.

EMBODIMENT 63. The apparatus of any preceding embodiment, wherein a surface area of the one or more electrodes is smaller than a surface area of the counter-electrode.

EMBODIMENT 64. The apparatus of any preceding embodiment, wherein the one or more electrodes comprises a plurality of individual disparate electrode surface areas formed in an array.

EMBODIMENT 65. The apparatus of any preceding embodiment, further comprising a power source for supplying an alternating current (AC) across the one or more electrodes and the counter-electrode.

EMBODIMENT 66. The apparatus of embodiment 65, wherein the AC is supplied at a voltage between about 1 mV and about 300 V.

EMBODIMENT 67. The apparatus of any preceding embodiment, wherein the AC is supplied at a voltage between about 1 mV and about 20 V.

EMBODIMENT 68. The apparatus of any preceding embodiment, wherein the AC is supplied at an oscillating frequency of between about 10 Hz and about 10 GHz.

EMBODIMENT 69. The apparatus of any preceding embodiment, wherein the AC is supplied at an oscillating frequency of between about 1 kHz and about 1 GHz.

EMBODIMENT 70. The apparatus of any preceding embodiment, wherein the one or more electrodes comprises at least one of a transparent conducting material or a doped semiconducting material.

EMBODIMENT 71. The apparatus of embodiment 70, wherein the transparent conducting material comprises indium tin oxide, graphene, doped graphene, a conducting polymer, or a thin metal layer.

EMBODIMENT 72. The apparatus of any preceding embodiment, wherein the one or more electrodes has a thickness between about 1 nm to about 50 μm.

EMBODIMENT 73. The apparatus of any preceding embodiment, wherein the one or more electrodes has a thickness between about 10 nm to about 5 μm.

EMBODIMENT 74. The apparatus of any preceding embodiment, wherein the fluid comprises one of an aqueous fluid, an aqueous buffer, an organic solvent, a hydrophobic fluid or a gas.

EMBODIMENT 75. The apparatus of any preceding embodiment, wherein the particle has a size between about 1 nm to about 1 mm.

EMBODIMENT 76. The apparatus of any preceding embodiment, wherein the particle comprises one of a biological organism, a biological structure, a cell, a living cell, viruses, oil droplets, liposomes, micelles, reverse micelles, protein aggregates, polymers, or surfactant assemblies.

EMBODIMENT 77. A method for operating an apparatus comprising providing a power source; providing one or more electrodes and a counter-electrode configured for generating a non-linear electric field for immobilizing a particle suspended in a fluid that flows between the one or more electrodes and the counter-electrode; providing a membrane disposed proximate a surface of the one or more electrodes, the surface of the one or more electrodes distal the counter-electrode, wherein the membrane is configured for separating the fluid from a compartment, and has an opening configured to allow for insertion of a sharp member disposed in the compartment; supplying, via the power source, an alternating current (AC) across the one or more electrodes and the counter-electrode, thereby generating an oscillating non-linear electric field; and immobilizing, via a dielectrophoretic force generated by the oscillating non-linear electric field, a particle suspended in the fluid.

EMBODIMENT 78. The method of embodiment 77, wherein the membrane comprises an opening.

EMBODIMENT 79. The method of any preceding embodiment, further comprising manipulating, via the opening, the particle that is immobilized.

EMBODIMENT 80. The method of any preceding embodiment, further comprising probing, via the opening, the particle with a sharp member configured to enter across the membrane from the compartment.

EMBODIMENT 81. The method of any preceding embodiment, further comprising inserting, via the opening, the particle with a sharp member configured to enter across the membrane from the compartment.

EMBODIMENT 82. The method of embodiment 81, wherein the sharp member comprises a Micro-Electro-Mechanical System (MEMS) structure or a Nano-Electro-Mechanical System (NEMS) structure.

EMBODIMENT 83. The method of any preceding embodiment, wherein the membrane comprises at least one of silicon nitride, silicon oxide, a metal oxide, a carbide, a ceramic, alumina, or a polymer.

EMBODIMENT 84. The method of any preceding embodiment, wherein the membrane has a thickness between about 10 nm to about 1 cm.

EMBODIMENT 85. The method of any preceding embodiment, wherein the membrane has a thickness between about 100 nm to about 10 μm.

EMBODIMENT 86. The method of any preceding embodiment, wherein the opening has a size between about 10 nm to about 50 μm.

EMBODIMENT 87. The method of any preceding embodiment, wherein the opening has a size between about 1 μm to about 5 μm.

EMBODIMENT 88. The method of any preceding embodiment, wherein a wall of the opening has a hydrophobic coating or a hydrophilic coating.

EMBODIMENT 89. The method of embodiment 88, wherein the hydrophobic coating has a contact angle between about 95° and about 165°.

EMBODIMENT 90. The method of any preceding embodiment, wherein the hydrophilic coating has a contact angle between about 20° and about 80°.

EMBODIMENT 91. The method of any preceding embodiment, wherein a surface area of the one or more electrodes is smaller than a surface area of the counter-electrode.

EMBODIMENT 92. The method of any preceding embodiment, wherein the one or more electrodes comprises a plurality of individual disparate electrode surface areas formed in an array.

EMBODIMENT 93. The method of any preceding embodiment, wherein the AC across the one or more electrodes and the counter-electrode is supplied at a voltage between about 1 mV and about 300 V.

EMBODIMENT 94. The method of any preceding embodiment, wherein the AC across the one or more electrodes and the counter-electrode is supplied at a voltage between about 1 mV and about 20 V.

EMBODIMENT 95. The method of any preceding embodiment, wherein the AC across the one or more electrodes and the counter-electrode is supplied at an oscillating frequency of between about 10 Hz and about 10 GHz.

EMBODIMENT 96. The method of any preceding embodiment, wherein the AC across the one or more electrodes and the counter-electrode is supplied at an oscillating frequency of between about 1 kHz and about 1 GHz.

EMBODIMENT 97. The method of any preceding embodiment, wherein the one or more electrodes comprises at least one of a transparent conducting material or a doped semiconducting material.

EMBODIMENT 98. The method of embodiment 97, wherein the transparent conducting material comprises indium tin oxide, graphene, doped graphene, a conducting polymer, or a thin metal layer.

EMBODIMENT 99. The method of any preceding embodiment, wherein the one or more electrodes has a thickness between about 1 nm to about 50 μm.

EMBODIMENT 100. The method of any preceding embodiment, wherein the one or more electrodes has a thickness between about 10 nm to about 5 μm.

EMBODIMENT 101. The method of any preceding embodiment, wherein the fluid comprises one of an aqueous fluid, an aqueous buffer, an organic solvent, a hydrophobic fluid or a gas.

EMBODIMENT 102. The method of embodiment 101, wherein the fluid is a first fluid, the compartment further comprises a second fluid, wherein the first fluid is a hydrophobic fluid and the second fluid is a hydrophilic fluid, or vice versa.

EMBODIMENT 103. The method of any preceding embodiment, wherein the first fluid and the second fluid are immiscible.

EMBODIMENT 104. The method of any preceding embodiment, wherein the particle has a size between about 1 nm to about 1 mm.

EMBODIMENT 105. The method of any preceding embodiment, wherein the particle comprises one of a biological organism, a biological structure, a cell, a living cell, viruses, oil droplets, liposomes, micelles, reverse micelles, protein aggregates, polymers, or surfactant assemblies.

EMBODIMENT 106. The apparatus of any preceding embodiment, wherein the fluid is a first fluid, the compartment comprises a second fluid that is immiscible with the first fluid.

EMBODIMENT 107. The method of any preceding embodiment, wherein the first fluid is a hydrophobic fluid and the second fluid is a hydrophilic fluid, or vice versa.

EMBODIMENT 108. The method of any preceding embodiment, wherein the fluid is a first fluid, the compartment comprises a second fluid that is immiscible with the first fluid.

EMBODIMENT 109. The method of embodiment 108, wherein the first fluid is a hydrophobic fluid and the second fluid is a hydrophilic fluid, or vice versa.

EMBODIMENT 110. The apparatus of any preceding embodiment, wherein the fluid is a first fluid, the compartment comprises a second fluid that is immiscible with the first fluid.

EMBODIMENT 111. The apparatus of embodiment 110, wherein the first fluid is a hydrophobic fluid and the second fluid is a hydrophilic fluid, or vice versa.

EMBODIMENT 112. A method for operating an apparatus comprising providing a power source; providing a membrane configured for separating a fluid from a compartment; providing a pair of electrodes disposed proximate a surface of the membrane, wherein the pair of electrodes is configured to generate a non-linear electric field across the electrodes; supplying, via the power source, an alternating current (AC) across the electrodes, thereby generating an oscillating non-linear electric field; and immobilizing, via a dielectrophoretic force generated by the oscillating non-linear electric field, a particle suspended in the fluid that flows between the electrodes.

EMBODIMENT 113. The method of embodiment 112, further comprising providing a counter-electrode, wherein the membrane comprises an opening.

EMBODIMENT 114. The method of any preceding embodiment, further comprising providing a third electrode disposed proximate the surface of the membrane.

EMBODIMENT 115. The method of any preceding embodiment, further comprising probing, via the opening, the particle with a sharp member configured to enter across the membrane from the compartment.

EMBODIMENT 116. The method of any preceding embodiment, further comprising: inserting, via the opening, the particle with a sharp member configured to enter across the membrane from the compartment.

EMBODIMENT 117. The method of any preceding embodiment, wherein each of the pair of electrodes comprises a sharp tip or flat tip, or the third electrode is a ring electrode.

EMBODIMENT 118. The method of any preceding embodiment, wherein the sharp member comprises a Micro-Electro-Mechanical System (MEMS) structure or a Nano-Electro-Mechanical System (NEMS) structure.

EMBODIMENT 119. The method of any preceding embodiment, wherein the membrane comprises at least one of silicon nitride, silicon oxide, a metal oxide, a carbide, a ceramic, alumina, or a polymer.

EMBODIMENT 120. The method of any preceding embodiment, wherein the membrane has a thickness between about 10 nm to about 1 cm.

EMBODIMENT 121. The method of any preceding embodiment, wherein the membrane has a thickness between about 100 nm to about 10 μm.

EMBODIMENT 122. The method of any preceding embodiment, wherein the opening has a size between about 10 nm to about 50 μm.

EMBODIMENT 123. The method of any preceding embodiment, wherein the opening has a size between about 1 μm to about 5 μm.

EMBODIMENT 124. The method of any preceding embodiment, wherein a wall of the opening has a hydrophobic coating or a hydrophilic coating.

EMBODIMENT 125. The method of any preceding embodiment, wherein the hydrophobic coating has a contact angle between about 95° and about 165°.

EMBODIMENT 126. The method of any preceding embodiment, wherein the hydrophilic coating has a contact angle between about 20° and about 80°.

EMBODIMENT 127. The method of any preceding embodiment, wherein the opening is disposed between the pair of electrodes.

EMBODIMENT 128. The method of any preceding embodiment, wherein the membrane comprises a plurality of electrode pairs formed in an array and a plurality of openings, wherein each of the openings is disposed between each of the plurality of electrode pairs.

EMBODIMENT 129. The method of any preceding embodiment, wherein the AC across the pair of electrodes and the counter-electrode is supplied at a voltage between about 1 mV and about 300 V.

EMBODIMENT 130. The method of any preceding embodiment, wherein the AC across the pair of electrodes and the counter-electrode is supplied at a voltage between about 1 mV and about 20 V.

EMBODIMENT 131. The method of any preceding embodiment, wherein the AC across the pair of electrodes and the counter-electrode is supplied at an oscillating frequency of between about 10 Hz and about 10 GHz.

EMBODIMENT 132. The method of any preceding embodiment, wherein the AC across the pair of electrodes and the counter-electrode is supplied at an oscillating frequency of between about 1 kHz and about 1 GHz.

EMBODIMENT 133. The method of any preceding embodiment, wherein one electrode of the pair of electrodes comprises at least one of a transparent conducting material or a doped semiconducting material.

EMBODIMENT 134. The method of embodiment 133, wherein the transparent conducting material comprises indium tin oxide, graphene, doped graphene, a conducting polymer, or a thin metal layer.

EMBODIMENT 135. The method of any preceding embodiment, wherein the pair of electrodes has a thickness between about 1 nm to about 50 μm.

EMBODIMENT 136. The method of any preceding embodiment, wherein the pair of electrodes has a thickness between about 10 nm to about 5 μm.

EMBODIMENT 137. The method of any preceding embodiment, wherein the fluid comprises one of an aqueous fluid, an aqueous buffer, an organic solvent, a hydrophobic fluid or a gas.

EMBODIMENT 138. The method of any preceding embodiment, wherein the particle has a size between about 1 nm to about 1 mm.

EMBODIMENT 139. The method of any preceding embodiment, wherein the particle comprises one of a biological organism, a biological structure, a cell, a living cell, viruses, oil droplets, liposomes, micelles, reverse micelles, protein aggregates, polymers, or surfactant assemblies.

EMBODIMENT 140. The method of any preceding embodiment, wherein the fluid is a first fluid, the compartment comprises a second fluid that is immiscible with the first fluid.

EMBODIMENT 141. The method of embodiment 140, wherein the first fluid is a hydrophobic fluid and the second fluid is a hydrophilic fluid, or vice versa.

EMBODIMENT 142. The apparatus of any preceding embodiment, wherein the one or more electrodes is disposed proximate to a surface of the membrane, the surface distal to the compartment.

EMBODIMENT 143. The apparatus of any preceding embodiment, wherein the one or more electrodes is disposed proximate to a surface of the membrane, the surface proximate to the compartment.

EMBODIMENT 144. The method of embodiment 26, wherein the one or more electrodes is disposed proximate to a surface of the membrane, the surface distal to the compartment.

EMBODIMENT 145. The method of embodiment 26, wherein the one or more electrodes is disposed proximate to a surface of the membrane, the surface proximate to the compartment.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

1. An apparatus comprising:
a membrane for separating a fluid from a compartment;
one or more electrodes disposed proximate to the membrane;
a counter-electrode,
wherein the one or more electrodes and the counter-electrode are configured to generate a non-linear electric field across the one or more electrodes and the counter-electrode; and
a power source for providing an alternating current (AC) across the one or more electrodes and the counter-electrode, thereby generating an oscillating non-linear electric field for immobilizing a particle suspended in the fluid that flows between the one or more electrodes and the counter-electrode.

2. The apparatus of claim 1, wherein the membrane comprises an opening.

3. The apparatus of claim 2, wherein the opening allows for mechanical manipulation of the particle that is immobilized and the mechanical manipulation includes probing the particle with a sharp member configured to enter across the membrane from the compartment.

4. The apparatus of claim 1, wherein the membrane comprises at least one of silicon nitride, silicon oxide, a metal oxide, a carbide, a ceramic, alumina, or a polymer.

5. The apparatus of claim 1, wherein the membrane has a thickness between about 10 mu to about 1 cm.

6. (canceled)

7. The apparatus of claim 2, wherein the opening has a size between about 10 nm to about 50 μm.

8. (canceled)

9. The apparatus of claim 2, wherein a wall of the opening has a hydrophobic coating or a hydrophilic coating.

10.-11. (canceled)

12. The apparatus of claim 1, wherein a surface area of the one or more electrodes is smaller than a surface area of the counter-electrode.

13.-15. (canceled)

16. The apparatus of claim 1, wherein the AC across the one or more electrodes and the counter-electrode is supplied at an oscillating frequency of between about 10 Hz and about 10 GHz.

17. (canceled)

18. The apparatus of claim 1, wherein the one or more electrodes comprises at least one of a transparent conducting material or a doped semiconducting material.

19. The apparatus of claim 18, wherein the transparent conducting material comprises indium tin oxide, graphene, doped graphene, a conducting polymer, or a thin metal layer.

20.-21. (canceled)

22. The apparatus of claim 1, wherein the fluid comprises one of an aqueous fluid, an aqueous buffer, an organic solvent, a hydrophobic fluid, or a gas.

23. (canceled)

24. The apparatus of claim 1, wherein the particle comprises one of a biological organism, a biological structure, a cell, a living cell, viruses, oil droplets, liposomes, micelles, reverse micelles, protein aggregates, polymers, or surfactant assemblies.

25. (canceled)

26. A method for operating an apparatus comprising:
providing a power source;
providing a membrane configured for separating a fluid from a compartment;
providing one or more electrodes disposed proximate to the membrane;
providing a counter-electrode,
wherein the one or more electrodes and the counter-electrode are configured to generate a non-linear electric field across the one or more electrodes and the counter-electrode;
supplying, via the power source, an alternating current (AC) across the one or more electrodes and the counter-electrode, thereby generating an oscillating non-linear electric field; and
immobilizing, via a di electrophoretic (DEP) force generated by the oscillating non-linear electric field, a particle suspended in the fluid that flows between the one or more electrodes and the counter-electrode.

27. The method of claim 26, wherein the membrane comprises an opening.

28. The method of claim 27, further comprising:
manipulating, via the opening, the particle that is immobilized.

29. The method of claim 27, further comprising:
probing, via the opening, the particle with a sharp member configured to enter across the membrane from the compartment.

30. The method of claim 27, further comprising:
inserting, via the opening, the particle with a sharp member configured to enter across the membrane from the compartment.

31.-52. (canceled)

53. An apparatus comprising:
one or more electrodes and a counter-electrode configured for generating a non-linear electric field for immobilizing a particle suspended in a fluid that flows between the one or more electrodes and the counter-electrode; and
a membrane disposed proximate a surface of the one or more electrodes, the surface of the one or more electrodes distal the counter-electrode,
wherein the membrane is configured for separating the fluid from a compartment, and has an opening configured to allow for insertion of a sharp member disposed in the compartment.

54.-75. (canceled)

76. The apparatus of claim 53, wherein the particle comprises one of a biological organism, a biological structure, a cell, a living cell, viruses, oil droplets, liposomes, micelles, reverse micelles, protein aggregates, polymers, or surfactant assemblies.

77.-145. (canceled)
